(19) United States
(12) Patent Application Publication
(10) Pub. No.: US 2019/0336520 A1
(43) Pub. Date: Nov. 7, 2019

(54) USE OF A SIRNA FOR TREATING CANCER

(71) Applicant: SELEXEL, Toulouse Cedex 1 (FR)

(72) Inventors: Florence CABON, GOYRANS (FR); Hilary BROOKS, SAINT ORENS DE GAMEVILLE (FR); Maud CHUSSEAU, SAUBENS (FR); Stéphanie DELMAS, FROUZINS (FR)

(21) Appl. No.: 16/345,586

(22) PCT Filed: Aug. 8, 2017
(86) PCT No.: PCT/FR2017/052214

§ 371 Date: Apr. 26, 2019

(30) Foreign Application Priority Data
Oct. 27, 2016 (FR) 1660439

(51) Int. Cl. A61K 31/713; A61P 35/00; A61K 38/18; A61K 38/39; C12N 15/113; A61M 5/142; C12Q 1/686; C12Q 1/6851

(52) U.S. Cl. CPC A61K 31/713; A61P 35/00; A61K 38/1866; A61K 38/1891; C12Q 1/6851; C12N 15/1138; A61M 5/142; C12Q 1/686; A61K 38/39

(57) ABSTRACT

Disclosed is a composition including at least one siRNA, the siRNA hybridising with a coding or non-coding mNRA, in which it induces degradation or inhibits translation, the expression of the mRNA or of the protein for which it codes being implicated in a pathology, for the use of same in the prevention and/or the treatment of the pathology, the composition being formulated for a continuous systemic administration mode, and a device including such an administration mode.

### BACKGROUND OF THE INVENTION

### Field of the Invention

The present invention relates to a new use of double-stranded oligonucleotides, and more particularly a use according to a new formulation and a new mode of administration.

### Description of the Related Art

Since the discovery of the RNA interference mechanism in 1998, considerable efforts have been made to develop the therapeutic potential of this tool for human diseases. Micro RNAs (miRNAs) are small RNAs encoded by the genome of all eukaryotic organisms. After transcription and maturation, they are loaded into a protein complex: RNA Induced Silencing Complex (RISC). When they hybridize with a messenger RNA (mRNA) or they induce its cleavage, leading to the degradation of the mRNA, or they inhibit its translation into protein. Interfering RNAs or Small Interfering RNAs (siRNAs) are synthetic double-stranded oligoribonucleotides that when introduced into cells mimic the action of miRNAs and trigger the RNA interference mechanism. Their mechanism of action is therefore not comparable to any other type of oligonucleotide.

A “double-stranded oligonucleotide” in the following refers more particularly to a siRNA. More precisely, when the double-stranded oligonucleotide is an siRNA, it is loaded into the RISC complex. One of the two strands, said “passenger” is cut and degraded, the other strand, said “guide”, remains in the RISC complex. This guide strand hybridizes with a region of an mRNA which it is complementary. This mRNA is called “siRNA target mRNA” and by extension, the gene that is transcribed to generate this RNA is called the “siRNA target gene”. This target mRNA can be coding or non-coding. An siRNA cleaves the target mRNA with which it hybridizes and causes its degradation, or it prevents its translation. This results in a decrease in the amount of the target mRNA, and/or in a decrease in the amount of protein encoded by the mRNA if it is a coding mRNA. These effects of an siRNA are collectively named in the following description “inhibitory effect of gene expression”.

The major obstacle that must be overcome in order to be able to use a siRNA for a therapeutic purpose, in particular in humans, is to obtain that the siRNA penetrates the tissue (s) of interest, that it stays there in an active state in sufficient concentration and long enough to produce the inhibition of the desired gene expression. The method of administration of siRNA should additionally be clinically acceptable, nontoxic and not trigger an adverse immune response. The method must be usable to deliver any type of siRNA, regardless of the siRNA target mRNA and its level of expression.

In the past, methods have been developed for delivering into the body other types of oligonucleotides such as, for example, antisense oligodeoxynucleotides (ODNs), ribozymes, aptamers, morpholinos, or triple helix oligonucleotides. Several of these oligonucleotides enter cells via a receptor-mediated mechanism of endocytosis (Vlassov et al, 1994), but such mechanisms have not been demonstrated for siRNAs. The methods developed for administering RNA or DNA single-stranded oligonucleotides, in particular in the absence of vectorization agents, are therefore not transposable to siRNAs and other methods have had to be developed for this purpose (Tatiparti et al, 2017).

Most therapeutic uses require that siRNA be administered systemically in the body. In what follows, systemic means that the siRNA is conveyed in the body to act at a distance from the place where it is administered, as opposed to a local or loco-regional administration, in particular as opposed to an intratumoral administration. The systemic distribution in the body is obtained by any method that results in a passage of siRNA in extracellular fluids such as blood, lymph or cerebrospinal fluid, that the compound containing the siRNA is ingested (orally), or injected (parenterally), or through the skin or mucous membranes.

Nucleic acids in general and siRNAs in particular are negatively charged. When they are outside the cells, this negative charge limits their penetration into the cells. For this reason, many methods, such as electroporation, liposomes, nanoparticles, polymers of different kinds, have been developed to make a siRNA penetrate into a cell in culture. However, these tools are not applicable to administer a siRNA systemically in a living organism. The negative charge of siRNAs facilitates their association with cationic molecules such as lipid or polymeric compositions. To use siRNAs in living organisms, siRNAs have been chemically conjugated or incorporated into different vectorization agents. A “vectorization agent” in the following refers to an agent which aims to convey the oligonucleotide in the biological fluids from the point of administration to the target tissue and to make it penetrate inside the body. the cell, either by penetrating the oligonucleotide into the interior of the cell, or by fusing with the plasma membrane of the cell and releasing the oligonucleotide therein.

These vectorization agents are on the one hand compounds containing macromolecules which form complexes with the oligonucleotides, in the form of a particle having a size greater than 20 nm, and on the other hand chemical conjugates associating via a link covalent one and/or the other strand of a siRNA to a compound intended to make it enter the cell, such as for example cholesterol or a penetrating peptide. “Penetrating peptides” are peptides capable of penetrating spontaneously inside the cells and retain this property when they are conjugated with a molecule, causing the crossing of the latter. Thus, the vectorization agents can be composed of different types of macromolecules, such as micellar lipids, cholesterol, liposomes, polymers, polyplexes, chitosans, quantum dots, penetrating peptides, dendrimers, derivatives of the polyethylenimine, nanoparticles, magnetic or super-magnetic spheres, or inorganic or organic nanostructures.

The most general consensus in the scientific and medical community is that non-vectorized siRNAs are ineffective, and that they must be stabilized and vectorized to be active (Scomparin et al., 2015). A large number of vectorization agents have thus been developed for siRNAs. Several are used in therapeutic trials in humans, in different indications. The implementation of these vectorization tools is generally complex, requiring successive steps and well-controlled processes and/or the covalent coupling of the oligonucleotide to another component. Several classes of vectorization agents, either because of their chemical or structural nature, or because of their association with an oligonucleotide, have been shown to exhibit toxic effects or trigger an undesirable immune response in animals or humans (Robbins et al, 2009), which is not the case with non-vectorized siRNAs (Heidel et al, 2004).

In addition, these vectorization agents often preferentially distribute the siRNAs in certain organs, in particular the liver, which limits their therapeutic use in other organs. An administration method that does not require the addition of vectorization agents is therefore advantageous.

Various methods have thus already been proposed to administer systemically non-vectorized siRNAs and to inhibit the expression of a gene in a tissue or in a tumor. The first proposed method, called hydrodynamic, was to inject intravenously a saline solution containing siRNA in seconds and in a large volume: 1.8 mL in the mouse, corresponding to more than half of its blood volume (McCaffrey et al., 2002). This method is inapplicable to humans and large animals.

Other authors have used an administration of non-vectorized siRNAs intraperitoneally in mice. This method is effective in inhibiting siRNA target expression, and inhibiting the growth of xenografted tumors in mice. This is illustrated in the literature (Delloye-Bourgeois et al., 2009, Pannequin et al, 2007). Intraperitoneal injection of siRNAs directed against the androgen receptor (siAR-1) (Compagno et al., 2007) or against thrombospondin-1 (siTSP1-1) (Firlej et al., 2011) effectively inhibits the growth of xenografted prostatic tumors in mice.

The intraperitoneal route is effective in animals but it is complex to use in humans, in particular if it must be used repeatedly, in particular because of infectious risks because it requires the surgical installation of a catheter and its use is generally restricted to the treatment of pathologies developing in the peritoneum or in intraperitoneal organs such as the ovaries. Even in these therapeutic indications, this route of administration presents significant obstacles which limit its use and effectiveness and it is therefore necessary to have alternative solutions (Zeimet et al., 2009).

The intravenous administration of non-vectorized siRNAs was also tested. However, like other oligonucleotides, intravenously injected siRNAs are eliminated by renal filtration (Van de Water et al., 2006).

Therefore, there is currently no siRNA mode of administration compatible with human clinical use, without a vectorization agent, making it possible to address them effectively in numerous target organs, in particular in the prostate, and/or in tumors and/or tumor metastases, for the purpose of preventing and/or treating pathologies. There is a real need to provide such a means. One of the aims of the invention is thus to provide modes of administration which make it possible to efficiently distribute siRNAs in numerous target organs, in particular in the prostate, and/or in tumors and/or in the metastases of these tumors, in order to prevent and/or treat pathologies resulting directly or indirectly from the expression of a gene, the siRNA targeting the mRNA transcribed from this gene.

Solutions have been described for targeting oligonucleotides to particular cells of the body. In what follows, an “addressing molecule” is a molecule addressing the oligonucleotide to a particular cell type, such as for example endothelial cells or cancer cells. An addressing molecule is not intended to penetrate the oligonucleotide inside the cell or to penetrate with the oligonucleotide but to increase its concentration in the outer membrane of the cell of interest. For example and non-exhaustively, an addressing molecule may be an aptamer, an antibody, transferrin, an RGD peptide, the ligand of a receptor, this addressing molecule interacting or binding to a molecule expressed at the targeted cell surface, such as a receptor, an integrin, a membrane antigen such as for example PSMA (Prostate Specifies Membrane Antigen).

The targeting molecule is usually either covalently coupled to the oligonucleotide, or incorporated into a vectorization agent, for example a nanoparticle or a liposome containing the oligonucleotide, so as to address the vectorization agent to the cell or the target tissue. There is no solution describing the mixture of a siRNA with a compound in a simple implementation, in particular without covalent coupling or without incorporation into a vectorization agent, and making it possible to address a siRNA towards a particular cell type.

The CD36 receptor is a membrane receptor expressed on the membrane of vascular and lymphatic endothelial cells and expressed by many types of cells, in particular tumor cells, for example leukemic cells. The CD36 receptor binds molecules of different natures. It is in particular a long-chain fatty acid receptor, in particular C16 or C18 fatty acids, an oxidized low-density lipoprotein receptor, or oxidized LDL receptor, an oxidized phospholipid receptor, a Thrombospondin receptor, a receptor of the hexarelin peptide, a fibril amyloid receptor.

The inhibitory effect of the gene expression of a siRNA is transient: when an siRNA enters a cell, it inhibits the expression of its target gene for a period that is shorter as the cells divide frequently, which is particularly the case of most cancer cells. The amount of mRNA transcribed from this target gene and/or the protein encoded by this mRNA is then restored, creating a “peaks and valleys” effect (Bartlett and Davis, 2006).

The effectiveness of an siRNA is dependent on its concentration in the cells of the targeted tissue and its residence time in that tissue. This concentration itself depends on the dose of siRNA administered, the stability of the latter in the extracellular media, its ability to penetrate the cells of the target tissues, the kinetics of this penetration, and that of its elimination. One of the aims of the invention is to provide methods of systemic administration of siRNA, and in particular formulations, which increase the concentration of siRNA in serum and/or tissues and/or prolong the duration of its effects. avoiding the effects of peaks and valleys.

### SUMMARY OF THE INVENTION

The invention thus relates to a composition comprising at least one siRNA, said siRNA hybridizing with a mRNA, coding or non-coding, of which it induces the degradation or of which it inhibits the translation, the expression of said mRNA or of the protein for which it being involved in a pathology, the composition being used for the prevention and/or treatment of said pathology, said composition being formulated for a continuous systemic mode of administration.

An siRNA according to the present invention is a pair of two oligoribonucleotides which hybridize with each other, each oligoribonucleotide comprising from 2 to 100, in particular 5 to

50, preferably 13 to 25 and more particularly 19, 20 or 21 ribonucleotides. “2 to 100” means 2, 3, 4, 5, 6, 7, 8, 9, 10, 11, 12, 13, 14, 15, 16, 17, 18, 19, 20, 21, 22, 23, 24, 25,
26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100.

According to the invention, and in a particular aspect, said siRNA may present chemical modifications such as chemical modifications on the guide strand or the passenger strand, on one or more nucleotides located at the 3′ or 5′ terminal ends, and/or on one or more nucleotides constituting the internal skeleton.

Said chemical modifications according to the invention are on ribose and/or base and/or phosphoric acid. Said chemical modifications according to the invention comprise at least one substitution of the 2′OH group of the ribose with a 2′-O-methyl RNA (2′OMe) or 2′-Y-methoxyethyl (2′MOE) group or 2′ fluoro (2F) or 2′-fluoro-β-arabinonucleotide (FANA), an allylation of 2′-oxygen to aminoethyl, guanidinoethyl-, cyanoethyl- or alkyl, replacement of the phosphodiester group by a phosphorothioate, a alkylation or thiolation of one or more nucleotides of siRNA, replacement of a ribonucleotide with a deoxyribonucleotide, or replacement of a nucleotide with a Locked Nucleic Acid (LNA).

In another particular aspect, said siRNA is devoid of chemical modification.

In a particular aspect, said siRNA is devoid of chemical modification, and comprises two deoxynucleotides overflowing at the 3′ end, in particular two deoxythymidines.

In another particular aspect, said siRNA is devoid of chemical modification, and does not comprise two deoxynucleotides overflowing at the 3′ end, in particular two deoxythymidines.

The invention relates to a composition for its above-mentioned use, wherein said siRNA can be any type of siRNA. Indeed, the formulation and the method of administration, object of the present invention, does not depend on the siRNA administered nor the siRNA target as illustrated by Examples 3 and 9.

In a particularly preferred aspect, the invention relates to a composition for its aforementioned use (systemic and continuous), wherein at least one siRNA comprises or consists of one of the pairs of oligonucleotides as defined in Table 1.

In a particular aspect, the invention relates to a composition for its above-mentioned use, wherein said at least one siRNA is one of the following siRNAs: siAR-1, siAR-1b, siAR-2, siAR-2b, siAR-3, siAR-3b, siAR-4, siAR4b, siAR-5, siAR-5b, siVEGF-1, siVEGF-1b, siTSP1-1, siTSP1-1b, siTSP1-2, siTSP1-2b, siTSP1-3, siTSP1-3b, siTSP1-4, siTSP1-4b, siTSP1-5, siTSP1-5b, siFoxP3-1, siFoxP3-1b, siFoxP3-2, siFoxP3-2b, as shown in Table 1, and SEQ ID NO: 1 to SEQ ID NO 52 sequences.

The present invention is also based on the unexpected results of the inventors who have discovered new siRNAs targeting the FoxP3 transcription factor.

The FoxP3 targeting siRNAs are more particularly used to target suppressive or immunosuppressive cells, particularly suppressor T cells, also called regulatory T cells, and in particular in all types of cancers or in autoimmune diseases. FoxP3 targeting oligonucleotides are also used to target cancer cells expressing this transcription factor.

In a particular aspect, the present invention also relates to a siRNA inhibiting the synthesis of the FoxP3 transcription factor, wherein said siRNA is one of the following siRNAs: siFoxP3-1, siFoxP3-1b, siFoxP3-2 or siFoxP3-2b such as defined in Table 1 for use as a medicament or for use in the prevention and/or treatment of a condition associated with the expression of the FoxP3 transcription factor in combination with a pharmaceutically acceptable carrier.

In a particular aspect, in said composition for its use in the prevention and/or treatment of a pathology associated with the expression of the FoxP3 transcription factor, said siRNA presents chemical modifications.

In a particular aspect, said composition for its use in the prevention and/or treatment of a pathology associated with the expression of the FoxP3 transcription factor, said siRNA is devoid of chemical modification.

In a particular aspect, said composition for use in the prevention and/or treatment of a pathology associated with the expression of the FoxP3 transcription factor, said siRNA is devoid of chemical modification and comprises two deoxynucleotides bridging at the 3′ end, including two deoxythymidines.

In a particular aspect, said composition for its use in the prevention and/or treatment of a pathology associated with the expression of the FoxP3 transcription factor, said siRNA is devoid of chemical modification and does not comprise two deoxynucleotides overflowing at the 3′ end, in particular two deoxythymidines.

As defined in Table 1, in the invention siRNA siAR-1, siAR-1b, siAR-2, siAR-2b, siAR-3, siAR-3b, siAR-4, siAR-4b, siAR-5, siAR-5b are collectively referred to as the siRNA-AR family.

As defined in Table 1, siVEGF-1, siVEGF-11 siRNAs are collectively referred to as the siRNA-VEGF family.

As defined in Table 1, in the invention, siRNA siTSPI-1, siTSP1b1, siTSP1-2, siTSP1-2b, siTSP1-3, siTSP1-3b, siTSP1-4, siTSP1-4b, siTSP1-5. siTSP1-5b are collectively referred to as the siRNA-TSP1 family.

As defined in Table 1, SiFOXP3-1, SiFOXP3-1b, SiFOXP3-2, siFOXP3-2b siRNA are collectively referred to as the siRNA-FoxP3 family.

The expression “at least 75% identity with a sequence” in Table 1 means 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%, in particular 79% %, 81%, 84%, 86%, 90%, 95% and 99%.

In a particular aspect, the invention relates to a composition for its above-mentioned use, wherein said at least one siRNA is a mixture of siRNA.

In a particular aspect, the invention relates to a composition for its above-mentioned use, wherein said mixture is a mixture of two siRNAs.

In a particular aspect, the invention relates to a composition for its above-mentioned use, wherein said mixture is a mixture of three siRNAs.

In a particular aspect, the invention relates to a composition for its above-mentioned use, wherein said siRNA mixture comprises or consists of the following siRNAs:
a siRNA belonging to the siRNA-AR family with a siRNA belonging to the siRNA-VEGF family, and in particular siAR-1 siRNA and siVEGF-1 siRNA;
a siRNA belonging to the siRNA-AR family with a siRNA belonging to the siRNA-TSP 1 family, and in particular siAR-1 siRNA and siTSP1-1 siRNA;
a siRNA belonging to the siRNA-AR family with a siRNA belonging to the siRNA-FoxP3 family, and in particular siAR-1 siRNA and siFoxP3-2 siRNA;
a siRNA belonging to the siRNA-VEGF family with a siRNA belonging to the siRNA-TSP1 family, and in particular siVEGF-1 siRNA and siTS1-1 siRNA;
a siRNA belonging to the siRNA-VEGF family with a siRNA belonging to the siRNA-FoxP3 family, and in particular the siVEGF-1 siRNA and the siFoxP3-2 siRNA;
a siRNA belonging to the siRNA-TSP1 family with a siRNA belonging to the siRNA-FoxP3 family, and in particular the siTSP1-1 siRNA and the siFoxP3-2 siRNA;
a siRNA belonging to the siRNA-VEGF family with a siRNA belonging to the siRNA-TSP1 family, and with a siRNA belonging to the siRNA-FoxP3 family, and in particular siVEGF-1 siRNA with the siTSPI-1 siRNA and with siFoxP3-2 siRNA; a siRNA belonging to the siRNA-VEGF family with a siRNA belonging to the siRNA-TSP1 family, with a siRNA belonging to the siRNA-AR family, and in particular siVEGF-1 siRNA with the siTSP1-1 siRNA and with the siAR-1 siRNA.

An advantageous aspect of the invention relates to a composition wherein said siRNA is siAR-1, of SEQ ID No. 1 and 2, for its use as a medicament or for its use for the prevention and/or treatment of an associated pathology to the expression of the androgen receptor, in particular for the prevention and/or treatment of prostate cancer or metastases of this cancer, in association with a pharmaceutically acceptable vehicle, according to a continuous systemic administration mode.

In all aspects of the present invention, the pathology according to the invention is a human or animal pathology.

According to one particular aspect of the invention, the pathology according to the invention is more particularly associated with the expression of the mRNA encoding the androgen receptor (AR), or the Thrombospondin-1 (TSP1), or the transcription factor. FoxP3, or the Vascular Endothelial Growth Factor A (VEGF).

The pathology according to the invention, whether or not associated with the expression of AR, or of TSP1, or of FoxP3, or of VEGF, is more particularly a primary tumor, a metastatic tumor, or a pathology associated with the presence of suppressive or immuno suppressive cells. A “primary tumor” according to the invention is in particular and without limitation a cancer of the anus, the appendix, the mouth, the bronchi and/or the upper airways, the bile duct, the nasal cavity and paranasal, brain, heart, cervix, colon, uterine body, stomach, liver, salivary glands, throat, tongue, lips, nasopharynx, esophagus, bones, ovary, pancreas, parathyroid, penis, pleura, lung, prostate, rectum, kidney, breast, adrenals, testes, head and neck, thymus, thyroid, urethra, vagina, gallbladder, bladder, vulva, gastrointestinal cancer, lymphoma, melanoma or cancer non-melanoma skin, myeloma, sarcoma, leukemia, mesothelioma, cholangiocarcinoma, osteosarcoma, glioblastoma, astrocytoma, oligodendroglioma, chondrosarcoma, a liposarcoma, a rhabdomyosarcoma, or a pheochromocytoma, collectively referred to as primary tumors in the following description, or the metastases of any of these primary tumors developing in other organs. Metastases represent a frequent and major complication of cancer and therapeutic failures in oncology are mainly related to the development of metastases. A primary tumor can disseminate to form one or more metastases, in one or more types of tissues such as bones, liver, spleen, ganglia or brain. In order to treat cancers with a siRNA or a combination of siRNA, it is therefore particularly useful and advantageous to have administration methods that distribute the siRNA (s) in several tissues.

The expression “pathology associated with the presence of suppressive or immunosuppressive cells” means that said suppressive or immunosuppressive cells facilitate the development of a pathology and in particular the initiation, implantation or development of a tumor or its metastatic dissemination. This term includes in particular regulatory T cells, also called T suppressors, Th17 lymphocytes and MDSCs (myeloid-derived suppressor cells).

In a particular aspect, the invention relates to a composition for its above-mentioned use, wherein said siRNA is used in combination with at least one anti-angiogenic agent and/or an anti-tumor agent and/or an immunotherapeutic agent, for use simultaneous, separate or spread over time. The term “separated or spread over time” also means “successive”.

According to the invention, the term anti-angiogenic agent means an agent for inhibiting the formation of blood vessels, in particular by inhibiting the expression or the function of VEGF, FGF2, PDGF, HGF, MET, FLT3, VEGFR1, VEGFR2, VEGFR3, KIT, TIE1, TIE2, RET, TRKB, AXL. Such agents are in particular Cilengitide, Vandetanib, Lenalidomide, Thalidomide, Arsenic Trioxide, Bevacizumab, or agents listed in Table 2.

According to the invention, an immunotherapeutic agent is an agent whose purpose is to stimulate, in particular by vaccination, and/or to restore an immune response, in particular by the inhibition of immunosuppressive or suppressive cells, and/or by inhibition of anergy of lymphocytes.

Immunotherapies within the meaning of the invention include therapies involving the administration of cytokines, antibodies targeting the control points and regulation of the immune system (immune checkpoint, for example PD1, PDL1, CTLA4, Tigit), treatment with T lymphocytes, genetically modified (Car-T) or not, or by dendritic cells, vaccination, antihelminth treatments. Such an agent is in particular chosen from Ipilimumab, nivolimumab, T-Vec, Sipuleucel-T, Blinatumomab and Pembrolizumab, or from the agents of Table 3.

According to the invention, an anti-tumor or chemotherapeutic agent is an agent possessing anti-cancerous properties chosen from: alkylating agents, anti-metabolite agents, cytotoxic antibiotics, topoisomerase I inhibitors, topoisomerase II inhibitors, anti-tumor antibiotics, genotoxic agents, PARP inhibitors, anti-microtubule agents. Such an agent is for example chosen from: Bendamustine, Temozolomide, Mechlorethamine, Cyclophosphamide, Carmustine, Cisplatin, Busulfan, Thiotepa, Decarbazine, Pentostatin, Methotrexate, Pemetrexed, Floxuridine, Fluorouracil, Cytarabine, Mercaptopurine, Thiguanine, Rubitecan, Mitomycin C, Daunorubicin, Doxorubicin, Bleomycin, Plicamycin, Mitoxantrone HCl, Oxaliplatin, Vinorelbine, BMS 184476, Vincristine sulfate, Vinblastine, Taxotere, Taxol, or the agents listed in Table 4.

In a particular aspect, the invention relates to a composition for its above-mentioned use, wherein said composition comprises a siRNA belonging to the siRNA-AR family in association with an anti-tumor agent and/or an immunotherapeutic agent and/or an agent antiangiogenic.

In a particular aspect, the invention relates to a composition for its above-mentioned use, wherein said composition comprises a siRNA belonging to the siRNA-VEGF family in association with an antitumor agent and/or an immunotherapeutic agent and/or an agent antiangiogenic.

In a particular aspect, the invention relates to a composition for its above-mentioned use, wherein said composition comprises a siRNA belonging to the siRNA-TSP1 family in association with an anti-tumor agent and/or an immunotherapeutic agent and/or an agent antiangiogenic.

In a particular aspect, the invention relates to a composition for its above-mentioned use, wherein said composition comprises a siRNA belonging to the siRNA-FoxP3 family in association with an anti-tumor agent and/or an immunotherapeutic agent and/or an agent antiangiogenic.

In all aspects of the present invention, said systemic administration mode according to the invention is chosen from the group comprising or consisting of one of the following modes of administration: subcutaneous, intraperitoneal, intravenous, intra-arterial, intracardiac, intramuscular, intradermal, intranasal, intravaginal, intrarectal, sublingual, oral, intrathecal, intraspinal, epidural, respiratory, cutaneous, transdermal, transmucosal.

In a particular aspect, the invention relates to a composition for its above-mentioned use, wherein said composition is formulated for a mode of administration at a therapeutically effective dose, and in particular at doses from 0.005 mg/kg/day to 30 mg/kg/day, in particular from 0.01 mg/kg/day at 10 mg/kg/day, and more particularly from 0.01 mg/kg/day to 2 mg/kg/day in humans.

From “0.005 mg/kg/day to 30 mg/kg/day” means all doses ranging from 0.005 mg/kg/day to 30 mg/kg/day, eg 0.008; 0.01; 0.05; 0.1; 0.5; 1.0; 1.5; 10.0; 10.5; 14.0; 14.5; 20; 20.5; 25; 25.5; 29.5 mg/kg/day.

In a first aspect, the present invention is based on the unexpected results of the Inventors that the efficacy of a siRNA administered by a continuous systemic mode of administration is better than when the same siRNA, formulated in the same solution, is administered by a bolus systemic administration mode, “in bolus” being defined as the administration of the full dose at one time, which dose may be repeated during treatment, for example every day or several times a day. The continuous mode of administration is defined as a mode that avoids the effects of peaks and valleys observed on siRNA concentration in the blood, serum and various organs when said siRNA is administered as a bolus. The purpose of continuous administration is to maintain substantially constant siRNA concentration in the blood and peripheral tissues throughout the siRNA administration period. The phrase “maintain substantially constant” means that the concentration of siRNA in the blood and peripheral tissues may vary slightly depending on the metabolism of the individual receiving said composition.

The administration time may vary from a few hours to several weeks depending on the device used to administer the siRNA. This device can be a pump or any composition for a slow release and prolonged release of siRNA.

In this particular aspect, the invention thus relates to a composition comprising at least one siRNA, said siRNA hybridizing with a mRNA, coding or non-coding, of which it induces the degradation or of which it inhibits the translation, the expression of said mRNA or of the protein for which it codes being involved in a pathology, the composition being used for the prevention and/or treatment of said pathology, said composition being formulated for a continuous systemic mode of administration.

In a preferred aspect of the invention, the siRNA administered in a continuous systemic mode of administration is administered without interruption of administration for a duration greater than or equal to 2 days. In a preferred aspect of the invention, the siRNA administered according to a continuous systemic mode of administration is administered without the administration being interrupted beyond the time necessary to recharge or exchange the device delivering the siRNA, for example 4 hours, for a period ranging from 2 days to 1 year, for example 2 days, 3 days, 4 days, 5 days, 6 days, 7 days, 8 days, 9 days, 10 days, 11 days, 12 days, 13 days, 14 days, 15 days, 3 weeks, 1 month, 2 months, 3 months, 4 months, 5 months, 6 months, 9 months, 1 year.

In a preferred aspect of the invention, siRNA administered in a continuous systemic mode of delivery is administered in successive cycles, interrupted by a period without treatment ranging from more than 24 hours to a few weeks, each cycle being defined by the administration. systemic continues without interruption greater than the time required to recharge or exchange the device delivering the siRNA, for example 4 hours, and for a period ranging from 2 days to 1 month.

In a preferred aspect according to the invention, said mode of continuous systemic administration is subcutaneous.

In a particular aspect, the invention relates to a composition for its above-mentioned use, wherein said composition is formulated for a mode of administration, continuously and subcutaneously, at a therapeutically effective dose, and in particular at doses from 0.005. mg/kg/day at 30 mg/kg/day, in particular from 0.01 mg/kg/day to 10 mg/kg/day and more particularly from 0.01 mg/kg/day to 2 mg/kg/day.

In a particular aspect, the invention relates to a composition for its above-mentioned use, wherein the sequence of one of the strands of said oligonucleotide is different from:

SEQ ID NO: 53
(GGGUUAUGUCUAUGUUCAUUCUU),
or
SEQ ID NO: 54
(GGAAUGGCCUGUGCUUUCUCAUU)

In a particular aspect, the invention relates to a composition for its above-mentioned use, wherein said continuous systemic mode of administration is one of the modes of administration. intraperitoneal, intravenous, intramuscular, intradermal, intranasal, intravaginal, intrarectal, sublingual, oral, intrathecal, AND said at least one siRNA belongs to one of the following four siRNA families: siRNA-AR family, siRNA-VEGF family, siRNA-TSP1 family, siRNA FoxP3 family.

In a particular aspect, the invention relates to a composition for its above-mentioned use, wherein said continuous systemic mode of administration is subcutaneous and said at least one siRNA belongs to one of the following four siRNA families: siRNA-AR family, siRNA-VEGF family, siRNA-TSP1 family, siRNA-FoxP3 family.

In a particular aspect, the invention relates to a composition for its above-mentioned use, wherein said pathology is any of the aforementioned primary tumors, or metastases of one of these primary tumors developing in other organs, and said at least one siRNA belongs to one of the following four siRNA families: siRNA-AR family, siRNA-VEGF family, siRNA-TSP1 family, FoxP3 siRNA family.

In a particular aspect, the invention relates to a composition for its aforementioned use, wherein said pathology is breast cancer, or melanoma, or glioblastoma, or kidney cancer, or liver cancer, or cancer of the liver. bladder, or cancer of the colon or metastases of one of these cancers developing in other organs, or leukemia or myeloma, and said at least one siRNA belongs to one of the following 4 siRNA families: siRNA-AR family, siRNA-VEGF family, siRNA-TSP1 family, FoxP3 siRNA family, and in particular siAR-1 siRNA, or siVEGF-1 siRNA, or siTS-1 siRNA, or siFoxP3-2 siRNA alone or in combination two to two or three to three.

In a particular aspect, the invention relates to a composition for its aforementioned use, wherein said pathology is a prostate cancer or metastases of this cancer developing in other organs, and said at least one siRNA belongs to the one of the following 4 siRNA families: siRNA-AR family, siRNA-VEGF family, siRNA-TSPI family, siRNA FoxP3 family, and in particular siAR-1 siRNA, siVEGF-1 siRNA, or siRNA siTSPI-1, or the siFNA siFoxP3-2, alone or in combination two by two or three to three and more particularly the siRNA siAR-1.

In another aspect, the invention also relates to a device providing a means of systemic and continuous administration of a composition formulated for a continuous systemic mode of administration comprising at least one siRNA said siRNA hybridizing with a mRNA encoding or not coding which it induces degradation or of which it inhibits translation, the expression of said mRNA or protein for which code said mRNA being involved in a pathology, and the composition being used for the prevention and/or treatment of said pathology.

In a particular aspect, said continuous systemic administration means is in particular an osmotic pump, a syringe pump, an elastomeric pump, a peristaltic pump, a multi-channel pump, a pump controlled by the patient, a “smart” pump, or a “patch” pump, or a polymeric matrix or a hydrogel, or any other biodegradable compound for slowly and continuously releasing the siRNA so that it is systemically distributed in the body. Some of these devices can be used in other therapeutic indications, to deliver a therapeutic agent discontinuously, in particular in bolus. In the present aspect, they are used to release the aforementioned composition with a substantially constant flow rate and continuously for several days to several weeks or more. The expression “substantially constant flow rate” means that the flow rate may vary slightly depending on the precision of the device used.

For example, the variation may be about plus or minus 10% with respect to the set flow rate.

The device can be mechanical or electronic. It can be worn outside or implanted surgically, for example under the skin, or in the peritoneum, or intramuscularly. In a non-exhaustive manner, these devices that can be used in the human clinic are: osmotic pumps, composed of a flexible reservoir surrounded by a compartment containing a saline gel which, by moisturizing, compresses the internal reservoir by forcing the expulsion of the liquid. This mechanism, which is used, for example, in Duras Durect pumps used in human clinics, is similar to that of Alzet pumps which are only authorized for use in animals;

automated syringes such as: McKinley T34 or T60, Bodyguard 323, AD syringe driver (Cardinal health), MS drivers (Smiths Medical); —elastomer pumps: the product is contained in a compressible reservoir contained in a balloon exerting a controlled pressure on the internal reservoir and forcing the expulsion of the liquid such as: Accufuser (WOO YOUNG Medical), Dosi-fuser (medical spirit), Exacta (Gamastech), Myfuser;
Peristaltic pumps: a flexible tubing is mechanically compressed to deliver the content such as: iPrecio, SP100 (APT instruments);
Multichannel or single channel pumps controlled by the patient, for example: Accucheck, one touch ping (Animas), Accufuser;
“patch” pumps: the product reservoir adheres directly to the skin and contains an integrated system, without tubing, of administration as for example: CeQurPaQ, Omnipod (Insulet), Finesse (Calibra), V-Go (Valeritas);
so-called “intelligent” pumps equipped with safety devices that modulate the administration of the product according to predefined parameters or measured in the patient, for example: miniMed 530G, paradigm Veo (medtronics), Vibe (Animas);
implantable pumps such as: Replenish minipump, Medtronic, Duras Durect, Infusaid, promedos. These pumps deliver small volumes of the therapeutic product at precise flow rates or automatically at predefined time intervals.

It can also be envisaged to put the dried oligonucleotides into a device that once put under the skin captures tissue water, and releases the siRNA that is solubilized in the extracellular fluids. It is also conceivable to incorporate the siRNA in a polymeric matrix, a gel or any other compound that releases the siRNA in a slow and prolonged manner over time. This device may be a mucosally adhering tablet and releasing the siRNA therethrough. In a particular aspect, said siRNA administered by the aforementioned device is associated with an addressing molecule.

In a particular aspect, said siRNA administered by the aforementioned device is not associated with an addressing molecule.

In a particular aspect, said at least one oligonucleotide which is administered by said device comprises or consists of one of the siRNAs belonging to the following siRNA families: siRNA-AR family, siRNA-VEGF family, siRNA-TSP1 family, siRNA FoxP3 family, and in particular siAR-1 siRNA, or siVEGF-1 siRNA, or siTSP-1 siRNA, or siFox-3-2 siRNA.

In a preferred aspect of the invention, siRNA administered in a continuous systemic mode of administration is diluted in an aqueous solution containing 154 mM NaCl.

In a preferred aspect of the invention, the siRNA administered according to a continuous subcutaneous systemic administration mode diluted in an aqueous solution containing 154 mM NaCl belongs to one of the following four siRNA families: siRNA-family AR, siRNA-VEGF family, siRNA-TSP1 family, FoxP3 siRNA family, and in particular siAR-1 siRNA, or siVEGF-1 siRNA, or siTPS1-1 siRNA, or siRNA siFoxP3-1, alone or in combination two to two or three to three.

In another aspect, the present invention is based on the unexpected results of the inventors, according to which the concentration of siRNA in serum, in many tissues and/or in tumors is higher when the siRNA is administered systemically while being formulated. in an acid pH buffer solution only when this same siRNA is formulated in an aqueous solution containing 154 mM NaCl. The presence of cations such as Zn2+ or Mg2+ in an aqueous solution of siRNA leads to their degradation and when siRNA is administered systemically, the presence of such cations in the injection solution reduces the concentration of siRNA in the serum and in the organs. Unexpectedly, the inventors have observed that the addition of cations in an acid pH buffer solution increases the concentration of siRNA in serum, in many tissues and/or in tumors, when said siRNA is administered systemically.

A buffer solution according to the present invention provides the pH stability of the siRNA dilution solution. Examples of such buffers are given in Table 5.

In this other aspect, the invention thus relates to a composition comprising at least one siRNA, said siRNA hybridizing with a mRNA, coding or non-coding, of which it induces the degradation or of which it inhibits the translation, the expression of said mRNA or of the protein for which it encodes being involved in a pathology, the composition being used for the prevention and/or treatment of said pathology, said composition being formulated for a continuous systemic mode of administration in which said at least one siRNA is in a buffer solution at acidic pH.

In a preferred aspect of the invention, the pH of the buffer solution is acidic, ranging from pH3 to pH 7, preferably from pH 5 to pH 6.5 and preferentially to pH 6.

In a preferred aspect of the invention the buffer solution is a citrate or histidine buffer at pH 6.

The present invention is also based on unexpected results of the inventors according to which the concentration of siRNA in serum, in many tissues and/or in tumors is higher when the siRNA is administered continuously systemically by being formulated in a buffer solution. at acidic pH containing cations from inorganic or organic salts, that when the same siRNA is formulated in an acidic buffer solution without cations or in a solution of 154 mM NaCl. These cations within the meaning of the invention are not constituents of the buffer solution, they are not intended to ensure a buffer effect, but they are added to this buffer solution. These cations are, for example and without limitation, polyamines, notably putrescine, and/or spermidine, and/or spermine, and/or salts whose cation is chosen from metal cations such as, for example, Zn2+, Co2+, Cu2+, Mn2+, Ca2+, Mg2+, Fe2+, the counterion being of any nature, for example a chloride, nitrate, sulfate or carbonate ion. In a preferred aspect of the invention, the buffer solution contains MgCl2, ZnCl2, MnCl2, or a two-by-two mixture of these salts, or a mixture of the three salts.

In a particular aspect, whether used alone or in combination, the concentration of each cation is from 0.02 mM to 200 mM, preferably from 0.05 to 100 mM and preferably from 1 to 50 mM. In a preferred aspect of the invention, the cations are added to a buffer solution which is a citrate buffer or a histidine buffer. In a preferred aspect of the invention, the pH of this solution is 6.

In a preferred aspect of the invention, the siRNA administered in a continuous systemic mode of administration is diluted in citrate or histidine buffer at pH 6 containing 10 mM MgCl2.

In a preferred aspect of the invention, the siRNA administered in a continuous systemic mode of administration, diluted in an acidic buffer containing cations, belongs to one of the 4 siRNA families: siRNA-AR family, siRNA-VEGF family, siRNA-TSP1 family, FoxP3 siRNA family, and particularly siAR-1 siRNA, siVEGF-1 siRNA, or siTSP1-1 siRNA, or siFoxP3-1 siRNA, alone or in combination two by two or three to three.

In another aspect, the present invention is based on the unexpected results of the inventors according to which the concentration of siRNA in serum, many tissues and/or tumors is higher when the siRNA is administered continuously systemically containing a molecule of addressing.

Thus, according to the invention and in a particular aspect, said composition contains an addressing agent, in particular, said composition contains an addressing agent not covalently coupled to siRNA.

According to the invention and in a particular aspect, said composition does not contain an addressing agent.

According to the invention and in a particular aspect, said composition is formulated for a continuous systemic mode of administration wherein said at least one siRNA is in an acidic pH buffer solution, said composition contains an addressing agent, in particular, said composition contains an addressing agent not covalently coupled to siRNA.

According to the invention and in a particular aspect, said composition is formulated for a continuous systemic mode of administration in which said at least one siRNA is in an acidic pH buffer solution, said composition does not contain an addressing agent.

In a particular aspect of the invention, this addressing molecule is a CD36 receptor ligand, for example oxidized LDLs, hexarelin or a long-chain fatty acid (more than 16 carbons), or a mixture of these. components two by two or three to three.

In a preferred aspect of the invention, the above-mentioned composition contains oxidized LDLs in a weight: weight ratio of 1 siRNA for 0.01 to 10 oxidized LDLs and preferentially 0.1 to 1, or hexarelin, in a weight: weight ratio of 1 siRNA for 0.01 to 10 hexarelin, preferably 0.1 to 1.

In a preferred aspect of the invention, the above-mentioned composition contains oxidized LDLs in a weight: weight ratio of 1 siRNA for 0.01 to 10 oxidized LDLs and preferentially 0.1 to 1, or hexarelin, in a weight: weight ratio. of 1 siRNA for 0.01 to 10 hexarelin, preferably 0.1 to 1 and is administered systemically continuously.

In a preferred aspect of the invention, the aforementioned composition is formulated for a continuous systemic mode of administration in which said at least one siRNA is in a buffer solution at acidic pH, in particular in a citrate or histidine buffer, and said composition contains an addressing agent not covalently coupled to siRNA, said addressing agent being a CD36 receptor ligand, said CD36 receptor ligand preferably being oxidized LDL, hexarelin, a long chain fatty acid, or a mixture of these components two by two or three to three.

In a preferred aspect of the invention, the aforementioned composition is formulated for a continuous systemic mode of administration wherein said at least one siRNA is in a citrate buffer and said composition contains an agent for addressing, said addressing agent being oxidized LDLs.

According to the invention, said composition comprises at least one siRNA, said siRNA hybridizing with a mRNA, coding or non-coding, of which it induces the degradation or of which it inhibits the translation, the expression of said mRNA or of the protein for which it being involved in a pathology code, the composition being used for the prevention and/or treatment of said pathology, said siRNA being in a solution containing or not containing vectorization agent.

In a particular aspect, the invention relates to an abovementioned composition comprising at least one siRNA, said siRNA being in a solution containing no targeting agent or wherein said siRNA is not associated with a vectorization agent.

In a particular aspect, the invention relates to an aforementioned composition comprising at least one siRNA, said siRNA being in a solution containing a vectorization agent.

In another aspect, the invention relates to a composition comprising at least one siRNA, said siRNA hybridizing with a mRNA, coding or non-coding, of which it induces the degradation or of which it inhibits the translation, the expression of said mRNA or the protein for which it codes being involved in a pathology, the composition being used for the prevention and/or treatment of said pathology, said composition being formulated for a systemic mode of administration other than a continuous mode of administration. Single or repeated bolus administration or slow infusion administration over a period of minutes to hours are non-limiting examples of a systemic mode of administration other than a continuous mode of administration.

In a particular aspect of the invention, systemic administration other than a continuous mode of administration of said composition comprising at least one siRNA can be associated with continuous systemic administration of said composition, simultaneously, separately or spread use over time.

In a particular aspect, said composition may be formulated for a systemic mode of administration other than a continuous mode of administration in which said at least one siRNA is in a buffer solution at acidic pH, in particular in a citrate or histidine buffer.

In a particular aspect of systemic administration other than a continuous mode of administration, the acidic pH buffer solution may be supplemented with inorganic or organic salts, in particular salts whose cation is chosen from polyamines, in particular chosen from spermine, spermidine or putrescine or in particular salt whose cation is chosen from metal cations, in particular chosen from salts of zinc, cobalt, copper, manganese, calcium, magnesium or iron, in particular of manganese, zinc, magnesium alone or in combination two to two or three to three

In a particular aspect of systemic administration other than a continuous mode of administration, said composition may contain an addressing agent.

In a particular aspect of systemic administration other than a continuous mode of administration, said composition contains an addressing agent, preferably not covalently coupled to siRNA.

In a particular aspect of systemic administration other than a continuous mode of administration, said composition does not contain an addressing agent.

Said addressing molecule may, for example, be a ligand of the CD36 receptor, such as oxidized LDL, hexarelin, a long chain fatty acid, or a mixture of these two to two or three to three component.

In a particular aspect, said siRNA is in a solution containing no vectorization agent or wherein said siRNA is not associated with a vectorization agent.

In a particular aspect, said siRNA is in a solution containing a vectorization agent.

In a particular aspect, the invention relates to a composition for use according to a systemic mode of administration, wherein said siRNA is used in combination with at least one anti-angiogenic agent and/or an anti-tumor agent and/or a immunotherapeutic agent, for simultaneous, separate or spread use over time.

In another aspect, the present invention is also based on the unexpected results of the inventors according to which the administration of siRNA targeting Thrombospondin-1 or VEGF, by a continuous mode of administration, intracerebral or intrathecal, also makes it possible to deliver these siRNAs effectively and thereby inhibit gene expression of the siRNA target gene.

Thus, and in this aspect, the present invention relates to a composition comprising at least one siRNA, said siRNA hybridizing with a coding or non-coding mRNA which it induces the degradation or of which it inhibits the translation, the expression of said mRNA or of the protein for which it encodes being involved in a pathology, the composition being used for the prevention and/or treatment of said pathology, said composition being formulated for a continuous, intracerebral or intrathecal mode of administration.

In one aspect of the invention, said composition for its aforementioned continuous and intracerebral use, is in particular used for the prevention and/or treatment of a brain cancer, in particular a glioblastoma. When the pathology is a brain cancer, in particular a glioblastoma, siRNAs are more particularly chosen from one of the following two siRNA families: siRNA-VEGF family, siRNA-TSP1 family, and in particular siVEGF-1 siRNA, or the siTSP1-1 siRNA, alone or in combination.

In a particular aspect of the invention, said composition for its above-mentioned use is formulated for a mode of administration at a therapeutically effective dose in continuous intracerebral or intrathecal, particularly at doses from 0.01 mg/kg/day to 10 mg/kg/day, in particular from 0.01 mg/kg/day to 2 mg/kg/day.

In another aspect, the invention relates to a pharmaceutical composition comprising as active substance at least one siRNA, said siRNA hybridizing with a mRNA, coding or non-coding, of which it induces degradation or of which it inhibits the translation, the expression of said mRNA or of the protein for which it encodes being involved in a pathology, said at least siRNA being in association with a pharmaceutically acceptable vehicle in a buffer solution at acid pH, in particular in a citrate or histidine buffer, with or without the addition of inorganic or organic salts, in particular of a salt whose cation is chosen from polyamines, in particular chosen from spermine, spermidine or putrescine or in particular salt whose cation is chosen from metal cations, in particular chosen from salts of zinc, cobalt, copper, manganese, calcium, magnesium or iron, in particular of manganese, zinc, magnesium, alone or in combination two to two or three to three.

In a particular aspect, said at least one siRNA is in a solution that does not contain a vectorization agent and is associated with an addressing molecule, or is in a solution containing a vectorization agent and is associated with a molecule of addressing, or is in a solution containing a vectorization agent and is not associated with an addressing molecule, or is in a solution that does not contain a vectorization agent and is not associated with an addressing molecule

TABLE 1
Oligonucleotide pairs constituting the siRNAs according to the present invention

| | | | SEQ | | |
|---|---|---|---|---|---|
| | | | ID | | spe- |
| Family | siRNA | compound | NO | Sequence 5′-3′ | cies |
| siRNA- | siAR-1 | siAR-1 strand 1, whose sequence is: SEQ ID NO: 1 | 1 | GACUCAGCUGCCCCAUCCA[dT][dT] | h, |
| AR | | or a sequence having at least 75% identity with | 2 | UGGAUGGGGCAGCUGAGUC[dT][dT] | r, |
| | | said SEQ ID NO: 1 and siAR-1 Strand 2, whose | | | so, |
| | | sequence is: SEQ ID NO: 2 or a sequence having | | | si, |
| | | at least 75% identity with said SEQ ID NO: 2 | | | c |
| | siAR-1b | siAR-1 strand 1b, whose sequence is: SEQ ID NO: 3 | 3 | GACUCAGCUGCCCCAUCCA | h, |
| | | or a sequence having at least 75% identity with | 4 | UGGAUGGGGCAGCUGAGUC | r, |
| | | said SEQ ID NO: 3and siAR-1 Strand 2b, whose | | | so, |
| | | sequence is: SEQ ID NO: 4 or a sequence having | | | si, |
| | | at least 75% identity with said SEQ ID NO: 4 | | | c |
| | siAR-2 | siAR-2 Strand 1, whose sequence is: SEQ ID NO: 5 | 5 | GACUCAGCUGCCCCAUCCACG[dT][dT] | h, |
| | | or a sequence having at least 75% identity with | 6 | CGUGGAUGGGGCAGCUGAGUC[dT][dT] | r, |
| | | said SEQ ID NO: 5 and siAR-2 Strand 2, whose | | | so, |
| | | sequence is: SEQ ID NO: 6 or a sequence having | | | si, |
| | | at least 75% identity with said SEQ ID NO: 6 | | | c |
| | siAR-2b | siAR-2 Strand 1b, whose sequence is: SEQ ID NO: 7 | 7 | GACUCAGCUGCCCCAUCCACG | h, |
| | | or a sequence having at least 75% identity with | 8 | CGUGGAUGGGGCAGCUGAGUC | r, |
| | | said SEQ ID NO: 7 and siAR-2 Strand 2b, whose | | | so, |
| | | sequence is: SEQ ID NO: 8 or a sequence having | | | si, |
| | | at least 75% identity with said SEQ ID NO: 8 | | | c |
| | siAR-3 | siAR-3 Strand 1, whose sequence is: SEQ ID NO: 9 | 9 | UCCCCAAGCCCAUCGUAGA[dT][dT] | h |
| | | or a sequence having at least 75% identity with | 10 | UCUACGAUGGGCUUGGGGA[dT][dT] | |
| | | said SEQ ID NO: 9 and siAR-3 Strand 2, whose | | | |
| | | sequence is: SEQ ID NO: 10 or a sequence having | | | |
| | | at least 75% identity with said SEQ ID NO: 10 | | | |
| | siAR-3b | siAR-3 Strand 1b, whose sequence is: SEQ ID NO: | 11 | UCCCCAAGCCCAUCGUAGA | h |
| | | 11 or a sequence having at least 75% identity | 12 | UCUACGAUGGGCUUGGGGA | |
| | | with said SEQ ID NO: 11 and siAR-3 Strand 2b, | | | |
| | | whose sequence is: SEQ ID NO: 12 or a sequence | | | |
| | | having at least 75% identity with said SEQ ID NO: | | | |
| | | 12 | | | |
| | siAR-4 | siAR-4 Strand 1, whose sequence is: SEQ ID NO: 13 | 13 | GUAGUUGUGAGUAUCAUGA[dT][dT] | h |
| | | or a sequence having at least 75% identity with | 14 | UCAUGAUACUCACAACUAC[dT][dT] | |
| | | said SEQ ID NO: 13 and siAR-4 Strand 2, whose | | | |
| | | sequence is: SEQ ID NO: 14 or a sequence having | | | |
| | | at least 75% identity with said SEQ ID NO: 14 | | | |
| | siAR-4b | siAR-4 Strand 1b, whose sequence is: SEQ ID NO: | 15 | GUAGUUGUGAGUAUCAUGA | h |
| | | 15 or a sequence having at least 75% identity | 16 | UCAUGAUACUCACAACUAC | |
| | | with said SEQ ID NO: 15 and siAR-4 Strand 2b, | | | |
| | | whose sequence is: SEQ ID NO: 16 or a sequence | | | |
| | | having at least 75% identity with said SEQ ID NO: | | | |
| | | 16 | | | |
| | siAR-5 | siAR-5 Strand 1, whose sequence is: SEQ ID NO: 17 | 17 | GCAUCAGUUCGCUUUUGAC[dT][dT] | h |
| | | or a sequence having at least 75% identity with | 18 | GUCAAAAGCGAACUGAUGC[dT][dT] | |
| | | said SEQ ID NO: 17 and siAR-5 Strand 2, whose | | | |
| | | sequence is: SEQ ID NO: 18 or a sequence having | | | |
| | | at least 75% identity with said SEQ ID NO: 18 | | | |
| | siAR-5b | siAR-5 Strand 1b, whose sequence is: SEQ ID NO: | 19 | GCAUCAGUUCGCUUUUGAC | h |
| | | 19 or a sequence having at least 75% identity | 20 | GUCAAAAGCGAACUGAUGC | |
| | | with said SEQ ID NO: 19 and siAR-5 Strand 2b, | | | |
| | | whose sequence is: SEQ ID NO: 20 or a sequence | | | |
| | | having at least 75% identity with said SEQ ID NO: | | | |
| | | 20 | | | |
| siRNA- | siVEGF- | siVEGF-1 Strand 1, whose sequence is: SEQ ID NO: | 21 | AUGUGAAUGCAGACCAAAGAA[dT][dT] | h, |
| VEGF | 1 | 21 or a sequence having at least 75% identity | 22 | UUCUUUGGUCUGCAUUCACAU[dT][dT] | r, |
| | | with said SEQ ID NO: 21 and siVEGF-1 Strand 2, | | | so, |
| | | whose sequence is: SEQ ID NO: 22 or a sequence | | | si, |
| | | having at least 75% identity with said SEQ ID NO: | | | c |
| | | 22 | | | |
| | siVEGF- | siVEGF-1 Strand 1b, whose sequence is: SEQ ID NO: | 23 | AUGUGAAUGCAGACCAAAGAA | h, |
| | 1b | 23 or a sequence having at least 75% identity | 24 | UUCUUUGGUCUGCAUUCACAU | r, |
| | | with said SEQ ID NO: 23 and siVEGF-1 Strand 2b, | | | so, |
| | | whose sequence is: SEQ ID NO: 24 or a sequence | | | si, |
| | | having at least 75% identity with said SEQ ID NO: | | | c |
| | | 24 | | | |
| siRNA- | siTSP1- | siTSP1-1 Strand 1, whose sequence is: SEQ ID NO: | 25 | CCUUGACAACAACGUGGUG[dT][dT] | h, |
| TSP1 | 1 | 25 or a sequence having at least 75% identity | 26 | CACCACGUUGUUGUCAAGG[dT][dT] | r, |
| | | with said SEQ ID NO: 25 and siTSP1-1 Strand 2, | | | so, |
| | | whose sequence is: SEQ ID NO: 26 or a sequence | | | si, |
| | | having at least 75% identity with said SEQ ID NO: | | | c |
| | | 26 | | | |
| | siTSP1- | siTSP1-1 Strand 1b, whose sequence is: SEQ ID NO: | 27 | CCUUGACAACAACGUGGUG | h, |
| | 1b | 27 or a sequence having at least 75% identity | 28 | CACCACGUUGUUGUCAAGG | r, |
| | | with said SEQ ID NO: 27 and siTSP1-1 Strand 2b, | | | so, |
| | | whose sequence is: SEQ ID NO: 28 or a sequence | | | si, |
| | | having at least 75% identity with said SEQ ID NO: | | | c |
| | | 28 | | | |
| | siTSP1- | siTSP1-2 Strand 1, whose sequence is: SEQ ID NO: | 29 | UACCCGAGACGAUUGUAUG[dT][dT] | h |
| | 2 | 29 or a sequence having at least 75% identity | 30 | CAUACAAUCGUCUCGGGUA[dT][dT] | |
| | | with said SEQ ID NO: 29 and siTSP1-2 Strand 2, | | | |
| | | whose sequence is: SEQ ID NO: 30 or a sequence | | | |
| | | having at least 75% identity with said SEQ ID NO: | | | |
| | | 30 | | | |
| | siTSP1- | siTSP1-2 Strand 1b, whose sequence is: SEQ ID NO: | 31 | UACCCGAGACGAUUGUAUG | h |
| | 2b | 31 or a sequence having at least 75% identity | 32 | CAUACAAUCGUCUCGGGUA | |
| | | with said SEQ ID NO: 31 and siTSP1-2 Strand 2b, | | | |
| | | whose sequence is: SEQ ID NO: 32 or a sequence | | | |
| | | having at least 75% identity with said SEQ ID NO: | | | |
| | | 32 | | | |
| | siTSP1- | siTSP1-3 Strand 1, whose sequence is: SEQ ID NO: | 33 | GCCAGAACUCGGUUACCAU[dT][dT] | so |
| | 3 | 33 or a sequence having at least 75% identity | 34 | AUGGUAACCGAGUUCUGGC[dT][dT] | |
| | | with said SEQ ID NO: 33 and siTSP1-3 Strand 2, | | | |
| | | whose sequence is: SEQ ID NO: 34 or a sequence | | | |
| | | having at least 75% identity with said SEQ ID NO: | | | |
| | | 34 | | | |
| | siTSP1- | siTSP1-3 Strand 1b, whose sequence is: SEQ ID NO: | 35 | GCCAGAACUCGGUUACCAU | so |
| | 3b | 35 or a sequence having at least 75% identity | 36 | AUGGUAACCGAGUUCUGGC | |
| | | with said SEQ ID NO: 35 and siTSP1-3 Strand 2b, | | | |
| | | whose sequence is: SEQ ID NO: 36 or a sequence | | | |
| | | having at least 75% identity with said SEQ ID NO: | | | |
| | | 36 | | | |
| | siTSP1- | siTSP1-4 Strand 1, whose sequence is: SEQ ID NO: | 37 | CCAACAAACAGGUGUGCAA[dT][dT] | h |
| | 4 | 37 or a sequence having at least 75% identity | 38 | UUGCACACCUGUUUGUUGG[dT][dT] | |
| | | with said SEQ ID NO: 37 and siTSP1-4 Strand 2, | | | |
| | | whose sequence is: SEQ ID NO: 38 or a sequence | | | |
| | | having at least 75% identity with said SEQ ID NO: | | | |
| | | 38 | | | |
| | siTSP1- | siTSP1-4 Strand 1b, whose sequence is: SEQ ID NO: | 39 | CCAACAAACAGGUGUGCAA | h |
| | 4b | 39 or a sequence having at least 75% identity | 40 | UUGCACACCUGUUUGUUGG | |
| | | with said SEQ ID NO: 39 and siTSP1-4 Strand 2b, | | | |
| | | whose sequence is: SEQ ID NO: 40 or a sequence | | | |
| | | having at least 75% identity with said SEQ ID NO: | | | |
| | | 40 | | | |
| | siTSP1- | siTSP1-5 Strand 1, whose sequence is: SEQ ID NO: | 41 | GCAACUACCUGGGUCACUA[dT][dT] | so |
| | 5 | 41 or a sequence having at least 75% identity | 42 | UAGUGACCCAGGUAGUUGC[dT][dT] | |
| | | with said SEQ ID NO: 41 and siTSP1-5 Strand 2, | | | |
| | | whose sequence is: SEQ ID NO: 42 or a sequence | | | |
| | | having at least 75% identity with said SEQ ID NO: | | | |
| | | 42 | | | |
| | siTSP1- | siTSP1-5 Strand 1b, whose sequence is: SEQ ID NO: | 43 | GCAACUACCUGGGUCACUA | so |
| | 5b | 43 or a sequence having at least 75% identity | 44 | UAGUGACCCAGGUAGUUGC | |
| | | with said SEQ ID NO: 43 and siTSP1-5 Strand 2b, | | | |
| | | whose sequence is: SEQ ID NO: 44 or a sequence | | | |
| | | having at least 75% identity with said SEQ ID NO: | | | |
| | | 44 | | | |
| siRNA- | siFoxP3- | siFoxP3-1 Strand 1, whose sequence is: SEQ ID NO: | 45 | CACAACAUGGACUACUUCA[dT][dT] | h, |
| FoxP3 | 1 | 45 or a sequence having at least 75% identity | 46 | UGAAGUAGUCCAUGUUGUG[dT][dT] | r, |
| | | with said SEQ ID NO: 45 and siFoxP3-1 Strand 2, | | | so, |
| | | whose sequence is: SEQ ID NO: 46 or a sequence | | | si, |
| | | having at least 75% identity with said SEQ ID NO: | | | c |
| | | 46 | | | |
| | siFoxP3- | siFoxP3-1 Strand 1b, whose sequence is: SEQ ID | 47 | CACAACAUGGACUACUUCA | h, |
| | 1b | NO: 47 or a sequence having at least 75% identity | 48 | UGAAGUAGUCCAUGUUGUG | r, |
| | | with said SEQ ID NO: 47 and siFoxP3-1 Strand 2b, | | | so, |
| | | whose sequence is: SEQ ID NO: 48 or a sequence | | | si, |
| | | having at least 75% identity with said SEQ ID NO: | | | c |
| | | 48 | | | |
| | siFoxP3- | siFoxP3-2 Strand 1, whose sequence is: SEQ ID NO: | 49 | CAUGGACUACUUCAAGUUC[dT][dT] | h, |
| | 2 | 49 or a sequence having at least 75% identity | 50 | GAACUUGAAGUAGUCCAUG[dT][dT] | r, |
| | | with said SEQ ID NO: 49 and siFoxP3-2 Strand 2, | | | so, |
| | | whose sequence is: SEQ ID NO: 50 or a sequence | | | si, |
| | | having at least 75% identity with said SEQ ID NO: | | | c |
| | | 50 | | | |
| | siFoxP3- | siFoxP3-2 Strand 1b, whose sequence is: SEQ ID | 51 | CAUGGACUACUUCAAGUUC | h, |
| | 2b | NO: 51 or a sequence having at least 75% identity | 52 | GAACUUGAAGUAGUCCAUG | r, |
| | | with said SEQ ID NO: 51 and siFoxP3-2 Strand 2b, | | | so, |
| | | whose sequence is: SEQ ID NO: 52 or a sequence | | | si, |
| | | having at least 75% identity with said SEQ ID NO: | | | c |
| | | 52 | | | |

For each sequence (SEQ ID NO) numbered from 1 to 52 of Table 1:
The first column indicates the family to which the siRNA belongs.
The second column indicates the name of the oligonucleotide.
The third column indicates the composition of the siRNA, constituted by the combination of a strand-type oligonucleotide (respectively 1b) or an oligonucleotide whose sequence has at least 75% identity with this strand-type oligonucleotide and strand-type oligonucleotide 2 (respectively 2b) or an oligonucleotide whose sequence has at least 75% identity with this strand-type oligonucleotide 2 (respectively 2b).
The fourth column indicates the numbering of the oligonucleotide as filed
The fifth column indicates the sequence in the 5′ to 3″ orientation. The notation [dT] [dT] is used to indicate the presence of two overhang deoxythymidines.
The sixth column indicates whether the siRNA constituted by the combination of the two oligonucleotides whose sequence is 100% identical to that indicated in column 5 hybridizes with human (h), rat (r), mouse (s), monkey (si) or dog (c) mRNA. Preservation of the sequence siRNA target between humans and other animal species is advantageous because it greatly increases the likelihood that the results of Preclinical studies, including toxicology studies, are predictive of human effects.

When the siRNAs shown in Table 1 consist of two single-stranded oligonucleotides whose sequence is 100% identical to those listed in the Table, these siRNAs furthermore have the following characteristics:
SiAR-2 siRNA is described in application PCT/FR2002/003843. The target sequence (i.e., the sequence of the mRNA to which the guide strand of this siRNA hybridizes) of this siRNA is present in humans in all mRNAs encoding the androgen receptor, that receptor is either wild-type, mutated, or has splicing variations leading to partial or complete deletion of the binding domain of hormones (varying AR-V7 for example). SiAR-1 siRNA corresponds to siAR-2 with 2 nucleotides deleted at the 3′ end.

The siRNA target sequences siAR-1, SiAR-1b, siAR-2, siAR-2b siRNA on the mRNA coding the androgen receptor are conserved in many species, including humans, rats, mice, monkeys and dogs.

The siRNA target sequences siAR-3 and SiAR-3b is located on the human androgen receptor-encoding mRNA. This target sequence is only partially conserved in other species.

The target sequence of siRNA siAR-4 and siAR-4b is located on the human mRNA coding for the variant V7 of the androgen receptor expressed in particular in castration-resistant prostate cancers. This target sequence is only partially conserved in other species.

The siRNA AR-5 is described in application PCT/FR2002/003843. The target sequence of this siRNA and the AR-5b siRNA is located in the human mRNA encoding the androgen receptor with the T877A mutation frequently found in prostate cancers. This target sequence is only partially conserved in other species.

SiVEGF-1 siRNA is described in patent application PCT/FR2002/003843. The target sequence of siVENA siVEGF-1, siVEGF-1b on the mRNA encoding VEGF is conserved in many species, including humans, rats, mice, monkeys and dogs.

SiTSP1-1, siTSP1b1, siTSP1-2 and siTSP1-2b have been described in application PCT/EP2010/061156. The siRNA siTSPI-1 siTSP1b1 target sequence on the Thrombospondin-1 mRNA is conserved in many species including humans, rats, mice, monkeys and dogs.
The target sequence of siRNA siTSP1-2 and siTSP1-2b on human mRNA encoding Thrombospondin-1 is partially conserved in other species.
The siRNA siTSP1-3, siTSP1-3b, siTSP1-4, siTSP1-4b, siTSP1-5, siTSP1-5b are the siRNAs corresponding to the siRNAs mentioned in the patent application US2011/0166199.
The siRNA siFOXP3-1, siFoxP3-1b, siFOXP3-2 and siFoxP3-2b are new and are described for the first time in this patent application. The target sequences of these siRNAs are located on the human mRNA encoding the FoxP3 transcription factor and are conserved in many species including humans, rats, mice, monkeys and dogs.

TABLE 2
Examples of anti-angiogenic agents (Drug name) that can be used in the present

| Drug name | Type | Mechanism of action | Clinical stage | Company |
|---|---|---|---|---|
| Bevacizumab | Humanized mAb | Blocks VEGF-A binding to | Approved for metastatic | Genentech/Roche (Basel, |
| (Avastin) | | receptors | CRC, NSCLC, RCC, recurrent | Switzerlanf) |
| | | | GBM | |
| Sunitinib | Small molecule | Inhibits signaling of VEGFRs, | Approved for metastic | Pfizer (New York, NY) |
| (Sutent) | RTK inhibitor | PDGFRs, FLT-3, CSF1R | RCC, imatinib-resistant | |
| | | | GIST, PNET | |
| Sorafenib | Small molecule | Inhibits signaling of VEGFRs, | Approved for metastic | Bayer/onyx (South San |
| (Nexavar) | RTK inhibitor | raf, PDGFRs, Kit | RCC, HPCC | Francisco, CA) |
| Pazopanib | Small molecule | Inhibits signaling of VEGFRs, | Approved for metastic RCC | Glaxo Smith Kline |
| (Votrient) | RTK inhibitor | PDGFRs, Kit | | (London, UK) |
| Vandetanib | Small molecule | Inhibits signaling of VEGFRs, | Approved for metastic | Astra Zeneca (London, |
| (Caprelsa) | RTK inhibitor | PDGFRs, EGFR | medullary thyroid cancer | UK) |
| Axitinib | Small molecule | Inhibits signaling of VEGFRs, | Approved for RCC that | Pfizer (New York, NY) |
| (Inlyta) | RTK inhibitor | PDGFRs, Kit | failed first-line therapy | |
| Aflibercept | Chimeric soluble | Binds VEGF-A, VEGF-B and | Phase 3 multiple tumor | Regeneron/Sanofi |
| | receptor | PLGF | types | Aventis (Paris) |
| AGM386 | Peptidobody | Binds Angiopoietin-1 and -2 | Phase 3 multiple tumor | Amgen(Thousand oaks, |
| | | | types | CA) |
| Motesanib | Small molecule | Inhibits signaling of VEGFRs, | Phase 3 multiple tumor | Amgen(Thousand oaks, |
| | RTK inhibitor | PDGFRs, Kit | types | CA) |
| Cediranib | Small molecule | Inhibits signaling of VEGFRs, | Phase 3 multiple tumor | Astra Zeneca (London, |
| (Recentin) | RTK inhibitor | PDGFRs, Kit | types | UK) |
| Cabozantinib | Small molecule | Inhibits signaling of VEGFRs, | Phase 3 multiple tumor | Exelixis (South San |
| | RTK inhibitor | PDGFRs, cMET, RET, Kit | types | Francisco, CA) |
| Tivozanib | Small molecule | Inhibits signaling of VEGFRs, | Phase 3 metastatic RCC | Aveo (Cambridge, MA) |
| | RTK inhibitor | PDGFRs, Kit | | |
| Regorafenib | Small molecule | Inhibits signaling of VEGFRs, | Phase 3 relapsed CRC and | Bayer/Onyx |
| | RTK inhibitor | PDGFRs, Kit | other umors | |
| Ramucirumab | Human mAb | Blocks VEGFR-2 signaling | Phase 3 multiple tumor | ImClone/Lilly |
| | | | types | (Indianapolis, IL) |
| Cilengitide | Cyclic peptide | Blocks αv integrins | Phase 3 GBM | Merck KGaA (Darmstadt, |
| | | | | Germany) |
| Volociximab | Chimeric mAb | Blocks α5β1 integrin | Phase 2 multiple tumor | PDL/Biogen Idec |
| | | | types | (Cambridge, MA) |
| IMC-18F1 | Human mAb | Blocks VEGFR-1 signaling | Phase 2 multiple tumor | ImClone/Lilly |
| | | | types | |
| TB-403 | Humanized mAb | Blocks PLGF binding to | Phase 2 multiple tumor | Thrombogenix/Roche |
| | | VEGFR-1 | types | |
| Anti-EGFL7 | Humanized mAb | Blocks EGFL7, a protein | Phase 2 multiple tumor | Genentech/Roche (Basel, |
| | | implicated in vascular | types | Switzerlanf) |
| | | maturation | | |

TKI, Tyrosine Kinase Inhibitor; CRC, Colorectal Cancer; NSCLC, Non-small cell lung carcinoma; RCC, renal cell carcinoma; GBM, Glioblastoma multiforme; GIST, gastrointestinal stromal tumor; HPCC, hepatocellular carcinoma

TABLE 3
Examples of Immunotherapeutic Agents (“Modality” Column) That Can Be Used in the Present Invention

| | | Pre-clinical | Pharmaco- | Pharmaco- | Clinical | |
|---|---|---|---|---|---|---|
| Modality | Status | findings | kinetics | dynamics | Efficacy | Safety |
| Cytokines | IL-2 and IFNα approved but | Moderate | Clear | Multiple effects, | Low | High unspecific, |
| | uncommonly used owing to high | effects | kinetics | MoA is complex | | toxicity (for |
| | toxicity and low efficacy | | | and hard to | | example, whole |
| | | | | attribute to | | body oedema) |
| | | | | one mechanism | | |
| Cellular | Multiple CAR-Ts and TCR-Ts in | Moderate- | In vivo | Clear MoA; target- | High response rates | Cytokine release |
| therapies | clinical trials; high complexity | to-strong | tracing and | dependent effects | depending on the | syndrome; target- |
| (CAR-Ts | of manufacture and supply chain; | effects | longevity of | | target (up to 90% | dependent cross- |
| and TCR-Ts) | strong target dependency; and | | infused cells | | for CD19, 50-60% for | reactivity with |
| | few clinically effective targets | | | | NY-ESO-1)129 | healthy tissue |
| | (for example, CD19 and | | | | | |
| | NY-ESO-1) | | | | | |
| Vaccines | Many types of cancer vaccines | Clear effects | No direct | Measurable immune | Minimal as | Minimal toxicity |
| | in clinical trials (including | in mice, but | pharmaco- | responses | monotherapy; | |
| | peptides, proteins, viruses | these do | kinetics | | combinations to be | |
| | and cells) | not directly | for peptide- | | explored | |
| | | translate | or protein- | | | |
| | | to humans | based | | | |
| | | | vaccines | | | |
| Checkpoint- | Ipilimumab (targeting CTLA4), | Moderate | Clear | Universal mechanism | Strong effects on | Distinct irAEs; |
| modulatory | pembrolizumab (targeting PD1) | effects | kinetics | not bound to | survival with long- | manageable with |
| antibodies | and nivolumab (targeting PD1) | | | histology, specific | term survival in a | treatment algorithms |
| | approved; many compounds | | | mutations or cancer | subset of patients | |
| | (including PDL1 blockers) in | | | antigens; multiple | | |
| | clinical investigation | | | downstream effects | | |
| | | | | after target | | |
| | | | | engagement | | |
| Connecting | Blinatumomab (BITE) approved | Strong in | Clear | Clear MoA; | High response rates | Moderate- to- |
| bi-specific | for CD19⁺ B cell malignancies | vitro cytolytic | kinetics | activating and | | severe toxicity |
| antibodies | | activity | | connecting T | | |
| | | | | cells to target- | | |
| | | | | expressing cancer | | |
| | | | | cells | | |
| Dual- | Multiple antibody formats in | Good effects | Clear | Dependent on | NA | NA |
| specific | discovery | (depending | kinetics | targets; dual | | |
| antibodies | | on the target) | | checkpoint | | |
| | | | | inhibition being | | |
| | | | | explored | | |
| Small | Several small molecules in | Strong effects | Clear | Clear on-target | Low as monotherapy; | Potential for off- |
| molecules | clinical trials (for example, | (depending on | kinetics | effects, with | combinations under | target toxicities |
| | targeting IDO) and multiple | the target) | | several targets | exploration | |
| | small molecules in discovery | | | located in | | |
| | | | | the tumour | | |
| | | | | microenvironment | | |
| Oncolytic | T-vec approved for unresectable | Moderate- | Clear | Systemic immune | Moderate response | Moderate toxicity |
| viruses | melanoma recurrent after initial | to-strong | kinetics | responses induced | rates as monotherapy; | for intra-tumoural |
| | surgery; several others under | effects | | by intra-tumoural | systemic effects after | injection |
| | clinical investigation, most | | | injection are | local injection | |
| | for intra-tumoural injection, | | | insufficiently | | |
| | with some expanding to systemic | | | studied | | |
| | administration | | | | | |
| Adjuvants | None approved, unsuccessfully | Anti-tumour | Clear | Multiple effects, | Low as monotherapy; | High toxicity if |
| | tested as monotherapies; new | effects in mice, | kinetics | MoA is complex | combination synergy | administered |
| | investigations now underway | particularly in | | and hard to | | systemically |
| | for combination therapies | combination* | | attribute to one | | |
| | | | | mechanism | | |

CAR-Ts, chimeric antigen receptor-transduced T cells; BITE, bi-specific T cell engager; IDO, indoleamine 2,3-dioxygenase; IFNα, interferon-α; IL-2, interleukin-2; irAE, immune-related adverse event; MoA, mechanisms of action; NA, not applicable; NY-ESO-1, cancer/testis antigen 1; TCR-Ts, T cell receptor-transduced T cells; T-vec, talimogene laherparepvec.
*In combination with with chemotherapy or checkpoint modulators

TABLE 4
Examples of anti-tumor agents (Drug Name) that can be used in the present invention

| Drug names (lead | | | Most recent reference |
|---|---|---|---|
| company) | Mode of action | Efficacy | or trial number |
| ARN-509 (Johnson & | Inhibits AR nuclear translocation | Reduction (≥50%) in PSA in ^{~}47% | Phase I²⁴⁹ |
| Johnson) | and DNA binding | patients at 12 weeks | Phase III ongoing²⁵⁰ |
| Cabometyx, Cometriq or | Small molecule inhibitor of FLT3, | COMET-1: median OS was 11.0 | Phase III²⁵¹ |
| cabozantinib (Exelis) | VEGFR1, VEGFR2, VEGFR3, c-Met, | months for drug versus 9.8 months | Combination studies |
| | KIT (also known as cKIT), TIE1, TIE2, | for prednisolone | ongoing²⁵² |
| | RET, TRKB and AXL | | |
| Custirsen or OGX-011 | Antisense oligodeoxynucleotide | SYNERGY: median OS of drug plus | Phase III²⁵³ |
| (Teva Pharmaceuticals) | that inhibits TRPM2 (also known as | docetaxel plus prednisone was 17 | Combination studies |
| | clusterin) | months, versus 14 months with | ongoing²⁵⁴ |
| | | docetaxel plus prednisone | |
| DCVAC/PCa (SOTIO | Dendritic cell vaccine against | Prolonged PSA-doubling time by ^{~}3.4 | Relapsed prostate |
| Group) | tumour antigens | times | cancer²⁵⁵ |
| | | | Phase III ongoing²⁵⁶ |
| Lutrate (GP Pharm) | Inhibits pituitary gland secretion of | Suppressed testosterone production | Phase III²⁵⁷ |
| | gonadotropins | in 97% of patients at 1 month | 3- and 6-month |
| | | | formulations are under |
| | | | development |
| Orteronel (Takeda) | Inhibits CYP17A1 and AR | Study terminated owing to lack of | Phase III²⁵⁸ |
| | | efficacy versus placebo | Maintenance studies |
| | | | suspended²⁵⁹ |
| ProstAtak or AdV-tk | Adenoviral vector expressing herpes | Reduced (20%) absolute risk in | Phase I²⁶⁰ |
| (Advantagene) | simplex virus thymidine kinase | recurrence for early-stage prostate | Phase III combination |
| | gene, plus synthetic acyclic | cancer | with radiotherapy |
| | guanosine analogue | | ongoing²⁶¹ |
| Prostvac or PSA-TRICOM | Vaccine vectors that infect antigen- | Improved median survival by ^{~}8.5 | Phase II¹⁷³ |
| (Bavarian Nordic) | presenting cells and generate | months and led to declines in PSA | Phase III ongoing²²² |
| | proteins for immune activation | (38%) and rate of PSA increase (47%) | |
| Sprycel or dasatinib | Inhibits SRC family protein tyrosine | No survival benefit in combination | Phase III combination |
| (Bristol-Myers Squibb) | kinases and BCR-ABL fusion protein | with docetaxel | with docetaxel²⁶² |
| TASQ (Active Biotech AB) | Inhibits angiogenesis by targeting | Improved PFS by 4 months versus | Phase II²⁶³ |
| | MRP14 | placebo in metastatic CRPC | Phase III completed²⁶⁴ |
| Yervoy or ipilimumab | CTLA4-specific antibody | Median OS was 11.2 months with | Phase III¹⁷⁰ |
| (Bristol-Myers Squibb) | | ipilimumab and 10.0 months with | |
| | | placebo | |
| DI17E6 or EMD525797 | Inhibits αV subunit of human | Radiographic SD at ≥18 weeks in 69% | Phase II²⁶⁵ |
| (Merck & Co.) | integrins | of patients | |
| Ozarelix (Spectrum | LHRH antagonist | Suppressed testosterone to castration | Preclinical²⁶⁶ |
| Pharmaceuticals) | | levels in hormone-dependent | Phase II completed²⁶⁷ |
| | | prostate cancer; reduced PSA | |
| | | by ≥50% in 97% of patients | |
| ATL101 (ATLAB Pharma) | Targets PSMA | Dose-dependent PSA decline of ≥50% | Phase II²⁶⁸ |
| | | in 10-27% of patients; CTC decline at | |
| | | 4-6 weeks in 64% patients | |
| BIND-014 (BIND | Polymeric nanoparticles targeting | Phase I: PR in one patient with | Phase I²⁶⁹ |
| Therapeutics) | PSMA and containing docetaxel | prostate cancer | Phase II completed²⁷⁰ |
| Capesaris or GTx-758 | Non-steroidal selective ER1α | PSA decline of ≥30% in 36% of | Phase II²⁷¹ |
| (GTx) | agonist | patients | |
| Quinacrine or CBLC102 | NF-κB transcription inhibition, p53 | Preclinical studies showed synergy | Preclinical²⁷² |
| (Cleveland BioLabs) | transcription induction (restoring | with paclitaxel; phase II data showed | Phase II completed²⁷³ |
| | p53-dependent apoptotic pathways | 1 of 31 patients had PR with | |
| | and tumour cell apoptosis) | quinacrine alone | |
| EPO906 or patupilone | Induces tubulin polymerization and | PSA decline of ≥50% in 13-47% of | Phase II²⁷⁴ |
| (Novartis] | stabilizes microtubules | patients; measurable PR in 24% of | |
| | | patients | |
| G-202 or thapsigargin | Targets PSMA and SERCA pump | Preclinical studies show ^{~}50% | Preclinical²⁷⁵ |
| prodrug (GenSpera) | | regression in mouse xenograft model | Phase II (withdrawn)²⁷⁶ |
| GVAX (Aduro Biotech) | Vaccine that expresses GM-CSF and | Stable PSA in 19% patients; ≥50% | Phase I/II²⁷⁷ |
| | stimulates the immune response | decline in PSA in 1 patient; median OS | |
| | | of 35 months in high-dose group | |
| IRX4204 (Io Therapeutics) | RXR agonist | PSA decline of ≥50% in 13% of | Phase II²⁷⁸ |
| | | patients | |
| ISIS-EIF4ERX (Isis | Antisense oligonucleotide that | Phase I: SD in 23% patients with | Phase I²⁷⁹ |
| Pharmaceuticals) | inhibits eIF4E | mixed tumours | Phase I/II combination¹⁰⁸ |
| Ixempra or ixabepilone | Promotes polymerization of tubulin | PSA decline of ≥50% in 17-48% of | Phase II²⁸⁰ |
| (Bristol-Myers Squibb) | and microtubule stabilization | patients; PR in 2-32% of patients | |
| KX2-391 (Kinex | SRC kinase inhibitor | PSA decline ≥30% in 10% of patients; | Phase II²⁸¹ |
| Pharmaceuticals) | | CTC conversion unfavourable-to- | |
| | | favourable in 18%; phase II study | |
| | | stopped early owing to pre-specified | |
| | | futility rule | |
| L-BLP25 (Merck & Co.) | Liposome-encapsulated peptide | PSA levels stable or reduced in 50% of | Phase II²⁸² |
| | vaccine containing MUC1-derived | patients with CSPC; PSA doubling time | |
| | antigen | prolonged by ≥50% in 38% of patients | |
| Olaratumab or LY3012207 | Blocks PDGFRα | Mixed tumour types: 63% had SD as | Phase I²⁸³ |
| (Eli Lilly and Company) | | best response | Phase II completed²⁸⁴ |
| ODM-201 (Bayer & Orion) | AR antagonist | PSA decline of ≥50% in 87% of | Phase I/II²⁸⁵ |
| | | patients; 100% SD or PR at 12 weeks | Phase III ongoing²⁸⁶ |
| OGX-427 (OncoGenex | Antisense oligonucleotide targeting | In combination with docetaxel, 2 of 7 | Phase I²⁸⁷ |
| Pharmaceuticals) | HSP27 | patients had PR and 1 of 7 prolonged | Phase II combination |
| | | SD; PSA decline of ≥30% in 30% of | ongoing²⁸⁸ |
| | | patients | |
| Lynparza or olaparib | PARP inhibitor | One of 3 patients (a BRCA mutation | Phase I²⁴⁰ |
| (AstraZeneca) | | carrier) with prostate cancer had PSA | Phase II ongoing²⁴² |
| | | decline of ≥50% | |
| Tigapotide or PCK3145 | Inhibits secretion of MMP9 and its | SD in 10 of 15 patients; PSA decline | Phase I²⁸⁹ |
| (Kotinos Pharmaceutical) | binding with CD44, interferes with | of ≥30% in 1 patient | |
| | VEGF signalling and reduces PTHRP | | |
| | levels | | |
| Danusertib or PHA- | Small-molecule inhibitor of AURKs | SD in 26% of patients; PSA decline | Phase II²⁹⁰ |
| 739358 (Nerviano Medical | | of ≥50% in 2 of 88 patients | |
| Sciences) | | | |
| PLX3397 (Daiichi Sankyo) | Inhibits FLT3, KIT, CSF1R | Reduction in CTCs in 3 of 4 patients | Phase I²⁹¹ |
| | | with mixed tumours | Phase II completed²⁹² |
| PSMA ADC (Progenics | Anti-PSMA monoclonal antibody- | PSA or CTC reduction in 50% of | Phase I²⁹³ |
| Pharmaceuticals) | MMAE conjugate | patients | Phase II completed²⁹⁴ |
| Ramucirumab (Eli Lilly and | Inhibits VEGFR2 | PR in 11% of patients with mixed | Phase I²⁹⁵ |
| Company) | | tumours; SD or PR in 30% of patients | |
| | | (1 of 2 with prostate cancer) lasting 6 | |
| | | months | |

ADC, antibody-drug conjugate; AR, androgen receptor; AURKs, Aurora kinases; CRPC, castration-resistant prostate cancer; CSF1R, macrophage colony-stimulating factor 1 receptor; CSPC, castration-sensitive prostate cancer; CTC, circulating tumour cell; CTLA4, cytotoxic T lymphocyte-associated antigen 4; CYP17A1, cytochrome P450 17A1; eIF4E, eukaryotic translation initiation factor 4E; ER1α, oestrogen receptor 1α; FLT3, Fms-like tyrosine kinase 3; GM-CSF, granulocyte-macrophage colony-stimulating factor; HSP27, heat shock protein 27; LHRH, luteinizing hormone-releasing hormone; MMAE, monomethyl auristatin E; MMP9, matrix metalloproteinase 9; MRP14, migration inhibitory factor-related protein 14; MUC1, mucin 1; NF-κB, nuclear factor-κB; OS, overall survival; PARP, poly(ADP-ribose) polymerase; PDGFRα, platelet-derived growth factor receptor-α; PFS, progression-free survival; PR, partial response; PSA, prostate-specific antigen; PSMA, prostate-specific membrane antigen; PTHRP, parathyroid hormone-related protein; RXR, retinoid X receptor; SD, stable disease; SERCA, sarcoplasmic/endoplasmic reticulum calcium ATPase; TIE, tyrosine kinase with immunoglobulin-like and EGF-like domains; TRKB, tropomyosin-related kinase B; TRPM2, testosterone-repressed prostate message 2; VEGF, vascular endothelial growth factor; VEGFR, VEGF receptor.

TABLE 5
Main buffers

| Buffer | pH | pKa 25° C. |
|---|---|---|
| maleate (pK1) | 1.2-2.6 | 1.97 |
| phosphate (pK1) | 1.7-2.9 | 2.15 |
| CABS | 10.0-11.4 | 10.7 |
| piperidine | 10.5-12.0 | 11.12 |
| glycine (pK1) | 2.2-3.6 | 2.35 |
| citrate (pK1) | 2.2-6.5 | 3.13 |
| glycylglycine (pK1) | 2.5-3.8 | 3.14 |
| malate (pK1) | 2.7-4.2 | 3.4 |
| formate | 3.0-4.5 | 3.75 |
| citrate (pK2) | 3.0-6.2 | 4.76 |
| succinate (pK1) | 3.2-5.2 | 4.21 |
| acetate | 3.6-5.6 | 4.76 |
| propionate | 3.8-5.6 | 4.87 |
| malate (pK2) | 4.0-6.0 | 5.13 |
| pyridine | 4.9-5.9 | 5.23 |
| piperazine (pK1) | 5.0-6.0 | 5.33 |
| cacodylate | 5.0-7.4 | 6.27 |
| succinate (pK2) | 5.5-6.5 | 5.64 |
| MES | 5.5-6.7 | 6.1 |
| citrate (pK3) | 5.5-7.2 | 6.4 |
| maleate (pK2) | 5.5-7.2 | 6.24 |
| histidine | 5.5-7.4 | 1.70, 6.04, |
| | | 9.09 |
| bis-tris | 5.8-7.2 | 6.46 |
| phosphate (pK2) | 5.8-8.0 | 7.2 |
| ethanolamine | 6.0-12.0 | 9.5 |
| ADA | 6.0-7.2 | 6.59 |
| carbonate (pK1) | 6.0-8.0 | 6.35 |
| ACES | 6.1-7.5 | 6.78 |
| PIPES | 6.1-7.5 | 6.76 |
| MOPSO | 6.2-7.6 | 6.87 |
| imidazole | 6.2-7.8 | 6.95 |
| BIS-TRIS propane | 6.3-9.5 | 6.80, 9.00 |
| BES | 6.4-7.8 | 7.09 |
| MOPS | 6.5-7.9 | 7.14 |
| HEPES | 6.8-8.2 | 7.48 |
| TES | 6.8-8.2 | 7.4 |
| MOBS | 6.9-8.3 | 7.6 |
| DIPSO | 7.0-8.2 | 7.52 |
| TAPSO | 7.0-8.2 | 7.61 |
| triethanolamine (TEA) | 7.0-8.3 | 7.76 |
| pyrophosphate | 7.0-9.0 | 0.91, 2.10, |
| | | 6.70, 9.32 |
| HEPPSO | 7.1-8.5 | 7.85 |
| POPSO | 7.2-8.5 | 7.78 |
| tricine | 7.4-8.8 | 8.05 |
| hydrazine | 7.5-10.0 | 8.1 |
| glycylglycine (pK2) | 7.5-8.9 | 8.25 |
| Trizma (tris) | 7.5-9.0 | 8.06 |
| EPPS, HEPPS | 7.6-8.6 | 8 |
| BICINE | 7.6-9.0 | 8.26 |
| HEPBS | 7.6-9.0 | 8.3 |
| TAPS | 7.7-9.1 | 8.4 |
| 2-amino-2-methyl-1,3-propanediol | 7.8-9.7 | 8.8 |
| (AMPD) | | |
| TABS | 8.2-9.6 | 8.9 |
| AMPSO | 8.3-9.7 | 9 |
| taurine (AES) | 8.4-9.6 | 9.06 |
| borate | 8.5-10.2 | 9.23, 12.74, |
| | | 13.80 |
| CHES | 8.6-10.0 | 9.5 |
| 2-amino-2-methyl-1-propanol | 8.7-10.4 | 9.69 |
| (AMP) | | |
| glycine (pK2) | 8.8-10.6 | 9.78 |
| ammonium hydroxide | 8.8-9.9 | 9.25 |
| CAPSO | 8.9-10.3 | 9.6 |
| carbonate (pK2) | 9.5-11.1 | 10.33 |
| methylamine | 9.5-11.5 | 10.66 |
| piperazine (pK2) | 9.5-9.8 | 9.73 |
| CAPS | 9.7-11.1 | 10.4 |
| phosphate (pK3) | | 12.33 |

### REFERENCES

Bartlett, D. W., and M. E. Davis. 2006. Insights into the kinetics of siRNA-mediated gene silencing from live-cell and live-animal bio luminescent imaging. Nucleic Acids Res 34: 322-333.
Compagno, D., C. Merle, A. Morin, Gilbert C., J. Mathieu, A. Bozec, C. Mauduit, M. Benhamed, and F. Cabon. 2007. SIRNA-Directed In Vivo Silencing of Androgen Receptor Inhibits the Growth of Castration-Resistant Prostate Carcinomas. PLoS ONE 2: e1006.
Delloye-Bourgeois, C., E. Brambilla, M. -Mr. Coissieux, C. Guenebeaud, R. Pedeux, V. Firlej, F. Cabon, C. Brambilla, P. Mehlen, and A. Bernet. 2009. Interference With Netrin-1 and Cell Death Tumor in Non-Small Cell Lung Cancer. J. Natl. Cancer Inst. 101:237-247.
Draper, D. E. 2004. A guide to ions and RNA structure. Rna 10: 335-343.
Firlej, V., J. R. R. Mathieu, C. Gilbert, L. Lemonnier, j. Nakhlé, C. Gallou-Kabani, B.
Guarmit, A. Morin, N. Prevarskaya, N B Delongchamps, and F. Cabon. 2011.
Thrombospondin-1 Triggers Cell Migration and Development of Advanced Prostate Tumors. Cancer Res earch 71: 7649-7658.
Forconi, M., and D. Herschlag. 2009. Metal Ion-Based RNA Cleavage as a Structural Probe. Methods in Enzymology 468: 91-106.
Heidel, J. D., S. Hu, X. F. Liu, T. J. Cheat, and M. E. Davis. 2004. Lack of interferon response in animals to naked siRNAs. Nat Biotechnol 22: 1579-1582.
McCaffrey, A. P., L. Meuse, T. T. Pham, D. S. Conklin, G. J. Hannon, and M. A. Kay. 2002. RNA interference in adult mice. Nature 418: 38-39.
Pannequin, J., N. Delaunay, M. Buchert, F. Surrel, J.-F. Bourgaux, J. Ryan, S. Boireau, J. Coelho, A. Pelegrin, P. Singh, A. Shulkes, M. Yim, G. S. Baldwin, C. Pignodel, G.
Lambeau, P. Jay, D. Joubert, and F. Holland. 2007. [beta]-Catenin/Tcf-4 Inhibition After Progastrin Targeting Reduces Growth and Drives Differentiation of Intestinal Tumors. Gastroenterology 133: 1554-1568.
Robbins, M., A. Judge, and I. MacLachlan. 2009. siRNA and Innate Immunity. Oligonucleotides 19: 89-102.
Scomparin, A., D. Polyak, A. Krivitsky, and R. Satchi-Fainaro. 2015. Achieving successful delivery of oligonucleotides—From physico-chemical characterization to in vivo evaluation. Biotechnology Advances 33: 1294-1309.
Tatiparti, K., S. Sau, S. K. Kashaw, and A. K. Iyer. 2017. siRNA Delivery Strategies: A Comprehensive Review of Recent Developments. Nanomaterials (Basel) 7:
van de Water, F. M., O. C. Boerman, A. C. Wouterse, J. G. P. Peters, F. G. M. Russel, and R. Masereeuw. 2006. Intravenously administered short interfering rna accumulates in the kidney and selectively suppresses gene function in renal proximal tubules. Drug metab set 34: 1393-1397.
Vlassov, V. V., L. A. Blakireva, and L. A. Yakubov. 1994. Of oligonucleotides across natural and model membranes. Biochimica and Biophysica Acta (BBA)—Reviews on Biomembranes 1197: 95-108. Zeimet, A. G., D. Reimer, A. C. Radl, A. Reinthaller, C. Schauer, E. Petru, N. Concin, S. Braun, and C. Marth. 2009. Pros and cons of intraperitoneal chemotherapy in the treatment of epithelial ovarian cancer. Anticancer Res 29: 2803-2808.

### BRIEF DESCRIPTION OF THE DRAWINGS

The invention will be better illustrated by the following examples and figures. The following examples are intended to clarify the object of the invention and illustrate advantageous embodiments, but in no case is intended to restrict the scope of the invention.

### Legend of Figures:

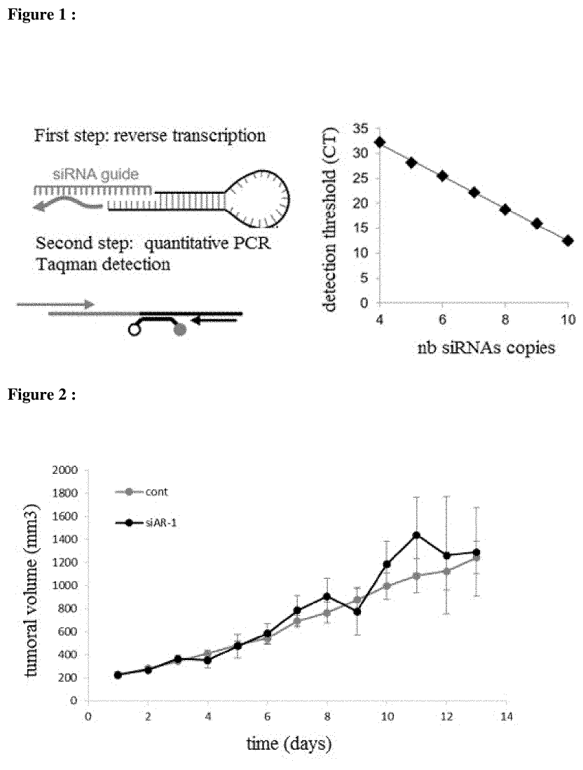

FIG. 1: Schematic representation of the method of quantification of a siRNA by RT-qPCR. Example of reference range.

FIG. 2: Intravenous injection of siAR-1 siRNA does not inhibit the growth of a prostate tumor xenografted in mice.

Growth of 22RV1 tumors xenografted in mice treated daily intravenously with a control siRNA (cont; gray curve) or with siAR-1 at 0.12 mg/kg (black curve). Mean±SEM, n=4 mice per group.

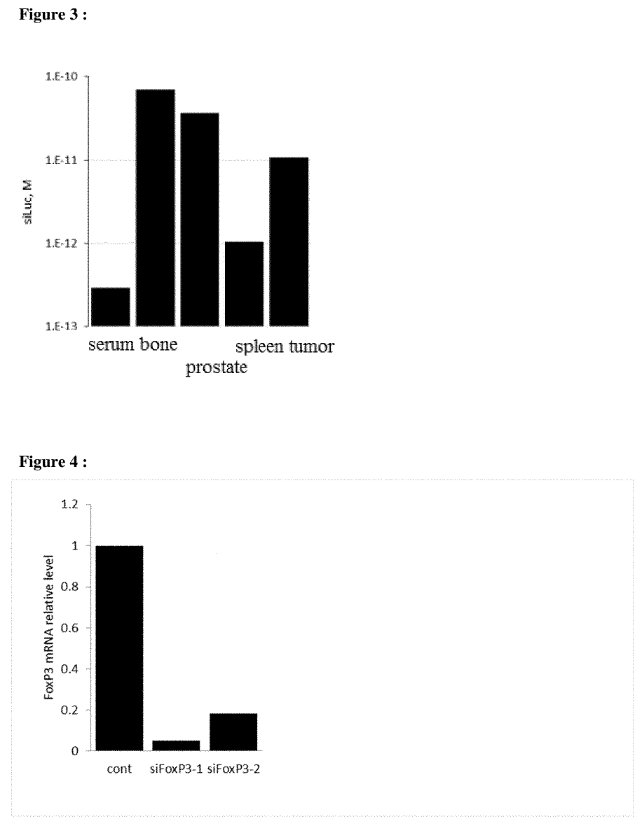

FIG. 3: Continuous systemic delivery of siRNA Luciferase (siLuc), which does not target mRNA expressed in mice, allows its distribution into serum and different organs.

Mean concentration (moles/L) of serum siRNA SiLuc in serum and various mouse organs (n=3) given by continuous subcutaneous administration of SiLuc for 3 days at 2 mg/kg/day diluted in NaCl solution 154 mM.

FIG. 4: FoxP3-1 and FoxP3-2 siRNAs inhibit FoxP3 expression in tumor cells. C4-2 prostate cells were transfected with siFoxP3-1 or siFoxP3-2 or with a control siRNA (cont). Two days after transfection, the amount of FoxP3 mRNA in the cells, relative to the amount of Cyclophilin A mRNA considered invariant (delta-delta CT method), was measured and compared to the value measured in the control condition.

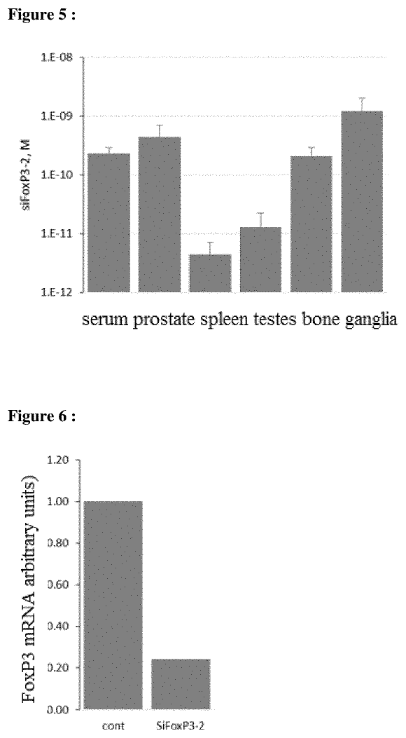

FIG. 5: siFoxP3-2 siRNA administered systemically subcutaneously is distributed in serum and various organs. Concentration (moles/L, mean±SEM, n=4 mice per group) in the serum and different organs of the siFoxP3-2 siRNA in mice administered daily for 4 consecutive days the siFoxP3-2 siRNA diluted in a citrate buffer containing 10 mM of MgCl2 administered at a dose of 0.12 mg/kg bolus subcutaneously.

FIG. 6: Daily administration of siFoxP3-2 siRNA for 3 consecutive days in male mice reduces the amount of FoxP3-encoding mRNA in the testes. Quantification of FoxP3-encoding mRNA, relative to the amount of mRNA encoding cyclophilin A in the testes of mice treated daily for 4 consecutive days in bolus subcutaneously with siRNA siFoxP3-2 diluted or in a citrate buffer containing 10 mM MgCl2, administered at a dose of 0.12 mg/kg (noted siFoxP3-2), or by the vehicle (noted “cont”).

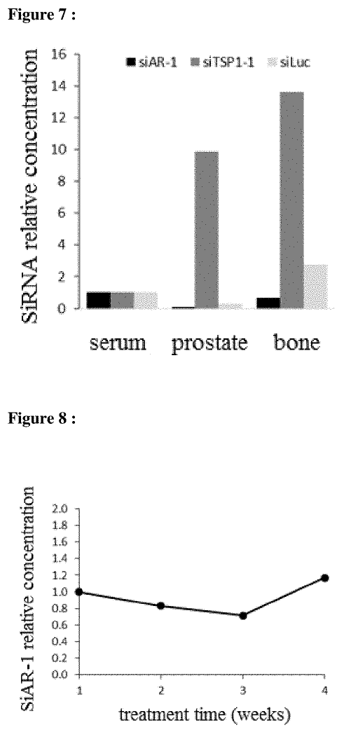

FIG. 7: The simultaneous administration of 3 siRNAs allows their distribution in serum and different organs. Concentration of siRNA siAR-1 (black bars), siTSP1-1 (dark gray bars) and siLuc (light gray bars) in serum, prostate and bone 10 minutes after subcutaneous injection of a mixture of these 3 siRNAs diluted in a 10 mM citrate buffer at pH 6 containing 10 mM MgCl2. For each siRNA, the serum concentration was considered as having a value of 1 and the organ concentrations were reported at this value in the serum.

FIG. 8: The continuous administration for 4 weeks of siAR-1 allows to maintain substantially constant its serum concentration in monkeys. Time concentration of siAR-1 formulated in 154 mM NaCl administered continuously subcutaneously for 4 weeks in monkeys (n=4) at a dose of 0.05 mg/kg/day. The mean serum concentration measured at the end of the second, third and fourth week of treatment is based on the average of that measured at the end of the first week of treatment.

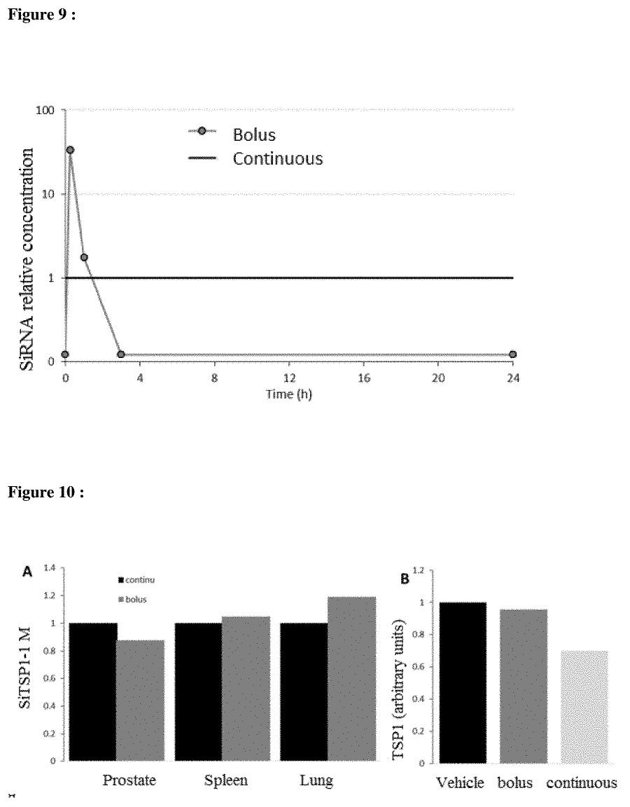

FIG. 9: Comparison of the pharmacokinetics in the serum of a siRNA after subcutaneous bolus administration or continuously.

Twenty-four hours prior to implantation of the osmotic pumps as described in FIG. 8, the same animals received a single bolus subcutaneous injection of 0.05 mg/kg of saline-formulated siAR-1 (154 mM NaCl) and the concentration was been measured over time. The solid line indicates the average of the values reported in FIG. 8. It is observed that the serum concentration of siRNA increases rapidly after the bolus injection and that the siRNA is rapidly eliminated, undetectable from the 3rd hour.

FIG. 10: Subcutaneous continuous systemic administration of a saline-formulated siRNA inhibits target expression in tissues more efficiently than with bolus administration.

Osmotic pumps delivering a siRNA dose siTSPI-1 of 0.12 mg/kg/day formulated in saline solution (154 mM NaCl) were implanted subcutaneously for 1 week in mice (“continuous” group, black bars). Another group of mice received a subcutaneous bolus injection of SiRNA TSP1-1 at a dose of 0.12 mg/kg of saline solution (bolus group, gray bars) for 7 days (n=5 mice per group). At the end of treatment, in each group, siRNA siTSP1-1 was quantified in different tissues (panel A) and mRNA encoding TSP1 measured in the prostate (panel B). In panel A, for each organ, the mean concentrations of siTSP1-1 was measured in the “bolus” group were reported as the average of those measured in the “continuous” group considered to have a value of 1. Panel B: The expression of the mRNA encoding TSP1 measured by RT-qPCR in the various organs and in the “continuous” (gray bars) or bolus (black bars) groups has been reported to that measured in the organs of a group of mice treated with 154 mM NaCl solution not containing siRNA (“vehicle” group, white bars).

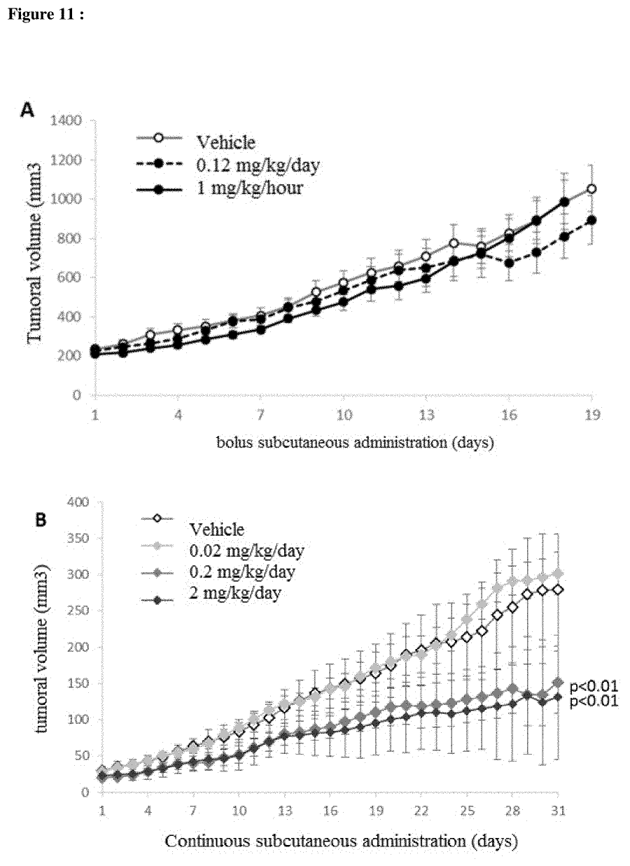

FIG. 11: Effect of bolus or continuous administration of siRNA on tumor growth. Nude mice were grafted with C4-2 prostate cells. Once the tumor was detected, the mice were treated with SiAR-1 siRNA.

Panel A: Measurement over time of tumor volume (mean±SEM, n=10 animals per group) in mice given siAR-1 formulated in saline (154 mM NaCl) and administered subcutaneously daily at the dose of 0.12 mg/kg/day (black symbols, discontinuous lines) or 1 mg/kg (black symbols, continuous lines). A control group (white symbols) received a daily injection of the vehicle (154 mM NaCl).

Panel B: Alzet implantable osmotic pumps were filled either with saline solution (vehicle group, 154 mM NaCl, white diamonds), or with siRNA siAR-1 formulated in saline (154 mM NaCl). The siRNA concentration was adjusted according to pump flow to deliver a daily dose of 0.02 mg/kg/day (light gray diamonds), 0.2 mg/kg/day (dark gray diamonds) or 2 mg/kg/day (black diamonds). The pumps were implanted subcutaneously in animals and tumors measured over time (mean±SEM, n=8 animals per group).

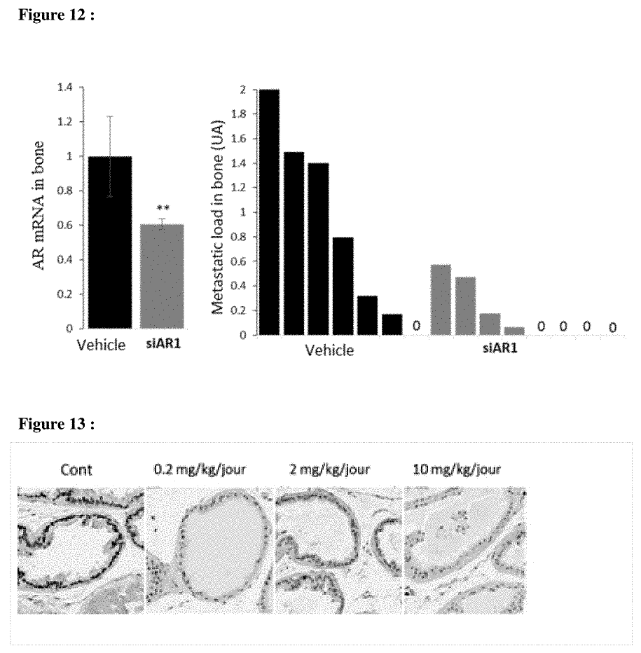

FIG. 12: Inhibition of bone metastases of prostate cancer by continuous systemic administration of siAR-1.

Left panel: Expression level of human AR mRNA in nude mouse tibia with 22RV1 human prostate tumors in mice treated with the vehicle (154 mM NaCl, black bar) or with diluted siAR-1 in this vehicle (gray bar) and administered subcutaneously continuously for 3 weeks (mean±SEM, n=7 values referred to the mean value of the NaCl group).

Right panel: Fsurre in bone as measured by the expression of human HPRT mRNA in both groups of animals. Each bar represents bone metastatic load in a mouse. “0” indicates that HPRT mRNA was not detected in this animal.

FIG. 13: Immunodetection of the androgen receptor in the prostate of mice treated with siAR-1 by continuous subcutaneous administration for 1 month. From left to right the photos are representative of the prostates of groups of mice (n=10) treated by the vehicle (154 mM NaCl), or siAR-1 at doses of 0.2 mg/kg/day, 2 mg/kg/day, or 10 mg/kg/day.

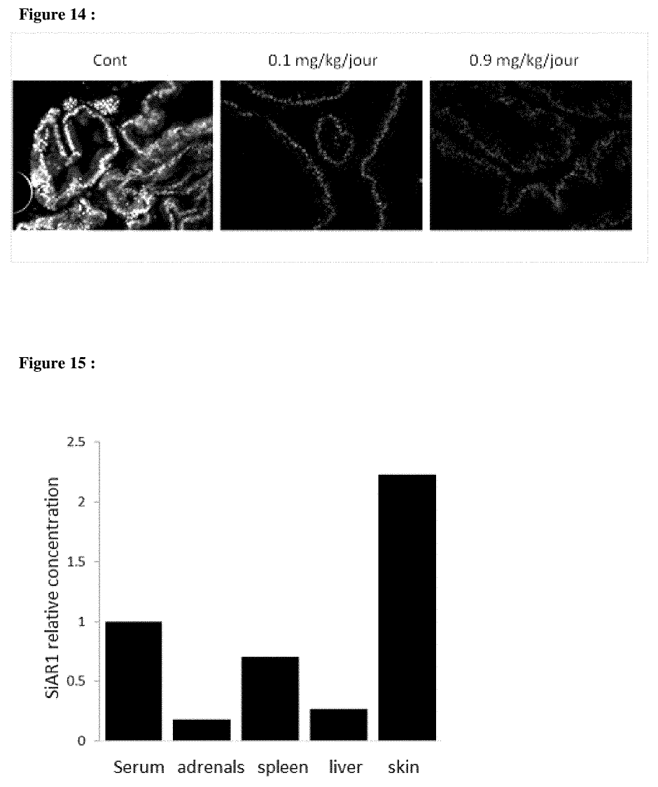

FIG. 14: Immunodetection of the androgen receptor in the prostate of rats treated with siAR-1 by continuous subcutaneous administration for 2 weeks. The photos are representative of vehicle-treated rat prostates (154 mM NaCl) “cont”, or siAR-1 at doses of 0.1 mg/kg/day or 0.9 mg/kg/day.

FIG. 15: Quantification of siAR-1 in various organs of monkeys that received continuous subcutaneous administration of this siRNA for one month at a dose of 5 mg/kg/day (n=4 animals). The concentration of siAR-1 in the organs was related to the measured concentration in the serum.

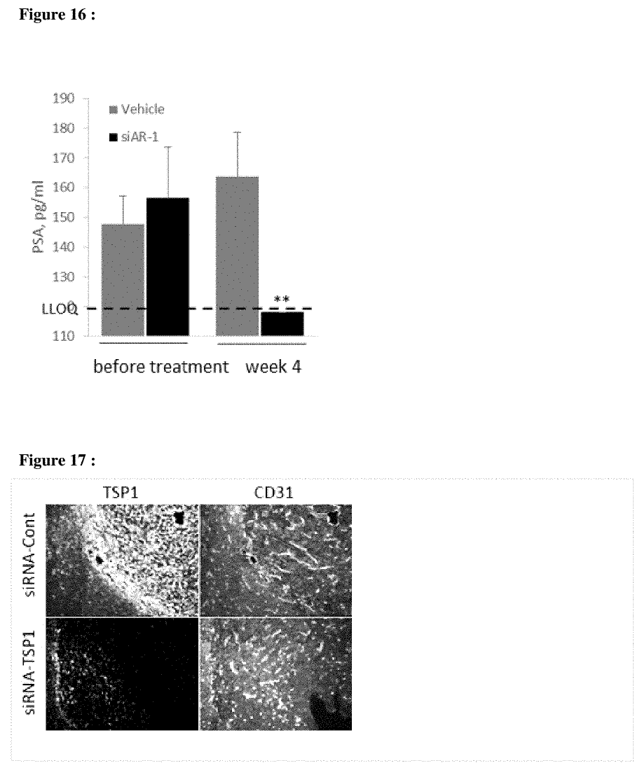

FIG. 16: Quantification of PSA (Prostate Specifies Antigen) in the serum of Cynomolgus monkeys (n=6 per group) before treatment, or after treatment for 1 month by continuous subcutaneous administration of saline (154 mM NaCl) or siAR-1 in saline at the dose of 5 mg/kg/day. Ordinate: blood PSA in pg/ml. The limit of detection of the ELISA (LLOQ or lower limit of quantification) indicated by a dotted line, is 120 pg/ml. Values below this value are arbitrarily indicated as 119 pg/ml.

FIG. 17: Immunodetection of TSP1 and blood vessels (CD31 staining) in U87 glioblastoma tumors implanted in nude mouse brain treated for 15 days by continuous intracerebral administration, of siTSP1-1 (noted siRNA TSP1) or a siRNA control (siRNA-cont), the catheter delivering the siRNA being implanted at a distance from the tumor.

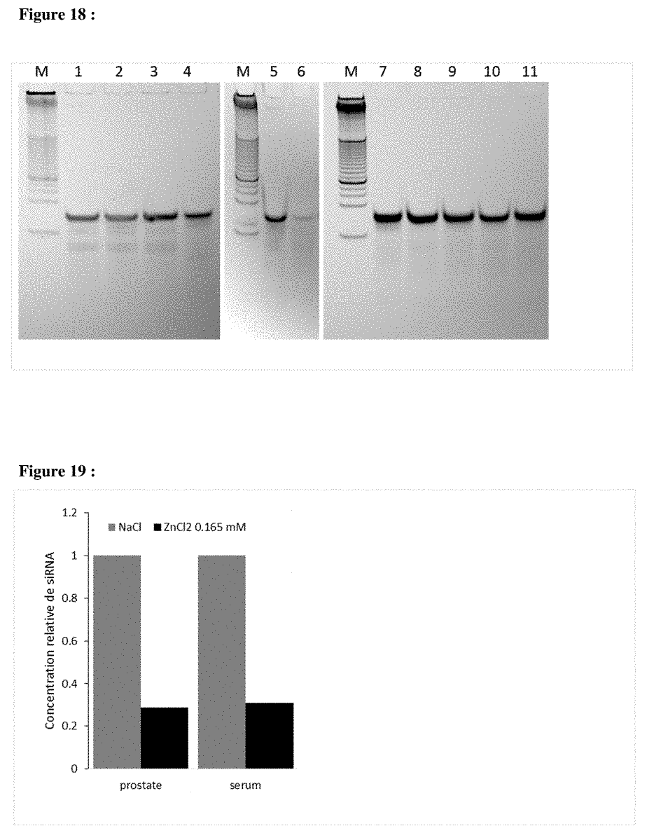

FIG. 18: Acrylamide gel electrophoresis analysis of the integrity of a siRNA incubated under different conditions. Lanes 1, 5, and 7: siTSP1-1 in aqueous solution; Lane 2: mixture (1:1, weight: weight) of oxidized SiTSP1-1 and LDL; Lane 3: mixture (1:10, weight: weight) of oxidized siTSP1-1 and LDL; Lane 4: mixture (1:1, weight: weight) of siTSP1-1 and hexarelin; Lane 6: siTSP1-1 in an aqueous solution adjusted to pH 6 containing 0.1 mM ZnCl2; Lane 8: siTSP1-1 in 10 mM citrate buffer pH 6; Lane 9-11: siTSP1-1 in 10 mM citrate buffer pH 6 supplemented with 1 mM ZnCl2 incubated at 37° C. for 10 minutes (lane 9), 1 hour (lane 10) or 6 h (lane 11). M: molecular weight marker (25 bp DNA ladder Invitrogen).

FIG. 19: Concentration of siTSP1-1 in the serum and prostate after administration of this siRNA subcutaneously bolus; Effect of the formulation.

Groups of adult mice were given a subcutaneous bolus injection of the SiTSP-1 siRNA at a dose of 0.12 mg/kg formulated either in a solution containing 154 mM NaCl (gray bar control group) or in an aqueous solution containing 0.165 mM ZnCl2 (black bars). The siRNA concentration measured in the serum and prostate of these different groups of mice was measured 20 minutes after injection and compared to the value of the control group considered as 1.

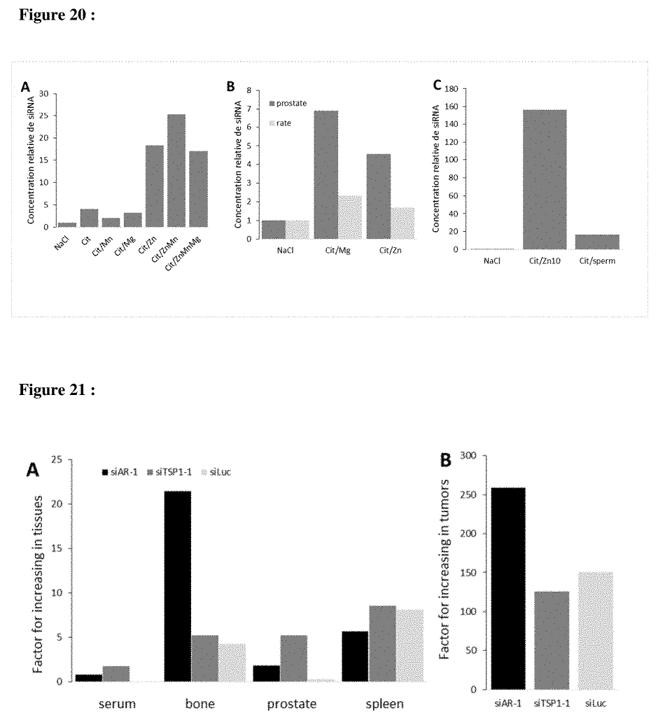

FIG. 20: Concentration of siAR-1 in serum and different organs depending on the formulation of the siRNA administered.

Concentration of siAR1 in serum or tissues of mice injected subcutaneously with siRNA siAR-1 formulated in one of the following solutions: 154 mM NaCl (NaCl, control group); 10 mM citrate buffer pH 6 (Cit); 10 mM pH 6 citrate buffer supplemented with 0.1 ml of MnCl2 (Cit/Mn), or 0.1 mM of MgCl2 (Cit/Mg), or 0.1 mM of ZnCl2 (Cit/Zn), or 0.1 ml of ZnCl2 and 0.1 mM MnCl2 (Cit/ZnMn), or 0.1 mM ZnCl2 and 0.1 mM MnCl2 and 0.1 mM MgCl2 (Cit/ZnMnMg), or 10 mM ZnCl2 (Cit/Zn10) or 0.05 mM spermidine (Cit/Sperm).

Panel A: Measured values in the serum of male animals given the indicated treatment.

Panel B: measured values in the prostate (dark gray bars) or spleen (light gray bars) of male animals given the indicated treatment.

Panel C: Measured values in the spleen of female animals that received the indicated treatment.

FIG. 21: A mixture of the following 3 siRNAs: siAR-1, siTSP1-1 and siLuc was prepared either in saline (154 mM NaCl) or 10 mM citrate buffer pH 6 containing 7.5 mM MgCl 2 and administered subcutaneously continued for 3 days to mice bearing 4T1 tumors, each siRNA being administered at a rate of 2 mg/kg/day. The siRNA has been assayed in serum and different organs, including 4T1 tumors. The concentrations of each siRNA, measured in serum, organs (panel A) and tumors (panel B), were reported to that measured for the siRNA considered in the serum of control group mice (154 mM NaCl). Black bars: siAR-1, dark gray bars, siTSP1-1, light gray bars: siLuc.

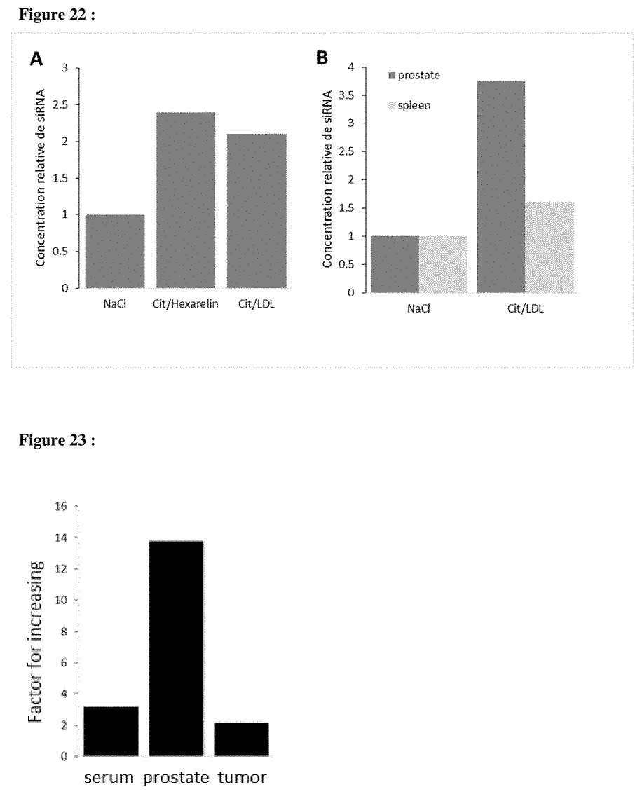

FIG. 22: siRNA siAR-1, at the dose of 0.12 mg/kg, formulated in a solution of 154 mM NaCl (control group, noted NaCl) or in a 10 mM citrate buffer pH 6 supplemented with either (siRNA: hexarelin, 1:0.2 weight: weight) Cit/Hexarelin), either oxidized LDL (siRNA: LDL oxidized, 1:1: weight: weight) (Cit/LDL) was administered subcutaneously to groups of adult mice. The concentration of siRNA measured in the serum (panel A), prostate (panel B, dark gray bars) or spleen (panel B, light gray bars) of these different groups of mice was measured 20 minutes after injection and reported to that of the control group (NaCl).

FIG. 23: The concentration in serum, tissues and tumors of a systemically administered and continuous subcutaneous siRNA is increased when formulated in citrate buffer containing oxidized LDL. Osmotic pumps were implanted subcutaneously into 22RV1 tumor-bearing mice in order to deliver systemically and continuously for 2 days 2 mg/kg/day of SiAR-1 and 0.2 mg/kg/day of oxidized LDL, formulated either in 154 mM NaCl is in 10 mM citrate buffer at pH 6. The concentrations of siAR-1 in serum, organs and tumors measured after injection of the citrate buffer composition were reported to those measured after injection of the NaCl composition.

### DESCRIPTION OF THE PREFERRED EMBODIMENTS

### Examples

### Example 1: Materials and Methods

1. Quantification of siRNA by a Modified Quantitative RT-PCR Method

To quantify a siRNA in the biological samples, the inventors have developed a reverse transcription method followed by a quantitative PCR (RT-qPCR).

For each siRNA, a specific stem-loop primer with 8 protruding nucleotides is synthesized, the 8 nucleotides being complementary to the 8 nucleotides of the 3′ end of the siRNA (antisense) guide strand. After a reverse transcription step, the product obtained is amplified by PCR using two primers, one hybridizing with the region corresponding to the loop of the reverse transcription primer. The 12 nucleotides at 3′ of the second primer having a DNA sequence corresponding to the 12 nucleotides of the 5′ end of the newly synthesized cDNA after reverse transcription of the siRNA guide strand. Detection of the amplification is carried out continuously by the degradation of a Taqman fluorescent probe or by incorporation of SybrGreen.

A range of double-stranded siRNAs, from 10³ to 10⁹ copies in the RT reaction, diluted in water is made and treated together with the samples by RT-qPCR. FIG. 1 shows schematically the RT-qPCR method and an exemplary range showing the relationship between the number of copies present in the reaction and the CT (cycle threshold or amplification threshold) obtained.

The biological samples in which siRNAs are quantified come from different sources:
Total RNAs extracted from known weight tissue fragments thought to contain siRNA. These RNAs are extracted by conventional methods such as by the phenol-chloroform method (trizol extraction). After extraction, they are diluted in water;
Serum. In this case, a known volume of serum is diluted in water containing an RNAse inhibitor at 1/100° or more diluted if the siRNA concentration is very high.

The CT values obtained for each sample are compared with those obtained for the range. This makes it possible to calculate the number of siRNA copies present in the assayed sample. The values are then reported firstly to the amount of total transcribed reverse RNA, then to the tissue weight from which the RNAs were extracted or to the serum volume, and the final results are expressed in moles/L (M) considering that the density of all tissues is 1 g/cm³.

2. Cell lines

The cell lines used in the examples are cell lines derived from prostate tumors in humans, resistant to castration and expressing the androgen receptor (lines C4-2 and 22RV1), mouse mammary tumors (4T1), or of human glioblastoma U87.

### 3. Tumor Cell Transplant in Mice

The tumors are obtained by subcutaneous injection of tumor cells into the flank of Nude mice (22RV1, C4-2 tumors) or BalB/C mice (4T1 tumors). Only animals on which tumor uptake is found are included in the study and randomized to receive treatment or control treatment. Bolus siRNA injections are given once a day, 5 days a week. In other experiments, U87 cells were implanted into the Nude mouse brain parenchyma by stereotaxic injection.

All siRNAs are diluted in water containing 154 mM NaCl or in the indicated buffer. When used, the osmotic pumps (eg Alzet pumps) are implanted subcutaneously on the back of the mice on the opposite side of the tumor if the mouse is wearing one. In the case of orthotopically implanted U87 tumors, the osmotic pump is implanted under the skin and a catheter placed at the outlet of the pump is connected to a device fixed on the cranial box by a cement and delivering the compound into the brain at distance of the previously implanted tumor.

The volume of subcutaneous tumors is estimated by measuring with a caliper the largest (D) and the smallest (d) diameter of the tumors. The volume is calculated by the formula V=D×d×d×0.5.

At the end of the experiment, the animals are sacrificed, the serum, the tumors and different tissues are dissected, the extracted RNAs and the siRNAs present in these RNAs are quantified.

All the experimental protocols used have been validated by the French ethics committees and regulatory authorities. They are implemented in such a way as to limit the number of animals used and to avoid unnecessary suffering.

4. siRNAs Used

The siRNAs used in the examples are those of Table 1.

In some experiments, a siRNA that does not hybridize with any known mRNA (siRNA Control) was used. The sequence of this siRNA is:

SEQ ID NO 55:
5′ UAGCAAUGACGAAUGCGUA [dT] [dT]
SEQ ID NO: 56:
5′ UACGCAUUCGUCAUUGCUA [dT] [dT]

A siRNA targeting luciferase, a gene that does not exist in mammals, has also been used. The sequence of this siRNA is:

SEQ ID NO. 57:
5′-CUUACGCUGAGUACUUCGA [dT] [dT]
SEQ ID NO 58:
5′-UCGAAGUACUCAGCGUAAG [dT] [dT]

### 5. Preparation of the Osmotic Pumps

The siRNAs are diluted in saline solution (water for injection with 154 mM NaCl) or in the indicated buffer at the concentration necessary to achieve administration of the desired amount over a period of 24 hours.

This concentration is calculated taking into account the hourly volume administered by the pump, as indicated by the manufacturer (Alzet). The pump is filled sterilely and implanted under the skin of treated animals (mice, rats, monkeys). The cathether placed at the outlet of the pump releases its contents either under the skin or in another location in order to obtain intrathecal or intracerebral administration. The pumps are held in place for a few days and up to 4 weeks according to the protocol indicated.

A preliminary study has shown that sterile solutions of siRNA are stable for at least 4 weeks at 37° C.

### Example 2 Absence of Inhibition of Tumor Growth by Intravenously Injected siRNAs in Bolus

9 week old male nude mice were subcutaneously xenografted on the flank with 22RV1 cells. After tumor initiation and randomization, the mice received a daily intravenous injection of siRNA control or siAR-1 at a dose of 0.12 mg/kg for 13 days. The tumor volume was measured daily. The results are shown in FIG. 2. It is found that IV injection of siAR-1 has no effect on tumor growth.

### Example 3

It is not necessary for siRNA to target mRNA expressed in the body for distribution. A siRNA directed against firefly luciferase siLuc, at a dose of 2 mg/kg/day, formulated in a 154 mM NaCl solution was administered to mice subcutaneously continuously for 3 days using implanted osmotic pumps. Quantification of the siRNA siLuc in the serum and various organs shown in FIG. 3 shows that it is efficiently distributed systemically even though there is no target mRNA of this siRNA in these tissues.

### Example 4: Identification of siRNA Inhibiting FoxP3 and Distributing in Tissues In Vivo

### 1. Inhibition of FoxP3 Transcription Factor Expression in C4-2 Cells.

C4-2 cells were transfected with a siRNA control or siFoxP3-1 siRNA or siFoxP3-2 siRNA. 48 hours after transfection, the cells were lysed, the extracted RNAs and FoxP3 expression measured by RT-qPCR. The values are normalized by the expression of the RNA encoding cyclophilin-A (delta delta CT method). The results are shown in FIG. 4 which shows that both FoxP3-1 and FoxP3-2 siRNAs inhibit FoxP3 expression.

2. The siFoxP3-2 siRNA is Distributed in Serum and Different Organs and Inhibits the Expression of FoxP3.

Mice (4 per group) received daily for 4 consecutive days subcutaneously or 0.12 mg/kg of siFoxP3-2 siRNA formulated in citrate buffer at pH 6 containing 10 mM MgCl2, or only this buffer (control group). FIG. 5 shows that siFoxP3-2 is systemically distributed in serum and in different organs, and FIG. 6 shows that in the testes, siFoxP3-2 strongly inhibits expression of FoxP3-encoding mRNA relative to the group. control.

Administered in the absence of a vectorization agent, the siFoxP3-2 siRNA is therefore capable of systemically distributing itself in vivo and of inhibiting the expression of its target gene.

### Example 5

Simultaneous administration of 3 siRNAs allows their systemic distribution in serum and various organs. The siRNA siAR-1, siTSP1-1 and siLuc were diluted in 10 mM citrate buffer at pH 6 containing 10 mM MgCl2. The mixture was administered subcutaneously to mice such that the mice received a dose of 0.12 mg/kg each of the 3 siRNAs. The mice were sacrificed 10 minutes after injection and each siRNA was assayed separately in serum and different tissues.

It is observed that the 3 siRNAs are present in the serum and in the various organs tested (FIG. 7). The administration of a cocktail of several siRNAs therefore allows their simultaneous systemic distribution in different tissues.

### Example 6

Administration of a siRNA formulated in saline solution subcutaneously continuous. In this example, the siRNAs are formulated in saline solution (154 mM NaCl).

1. The Serum Concentration of a Continuously Administered siRNA Remains Substantially Constant.

Osmotic pumps delivering a dose of siAR-1 siRNA of 0.05 mg/kg/day formulated in 154 mM NaCl solution were implanted subcutaneously for 4 weeks in Cynomolgus monkeys. The serum siRNA concentration was measured weekly for the duration of the treatment. It is observed that the serum concentration varies by less than 20% compared to the first measurement (considered to have the value 1) (FIG. 8).

Twenty-four hours prior to implantation of the osmotic pumps, the animals received a single bolus subcutaneous injection of 0.05 mg/kg of saline formulated siAR-1 (154 mM NaCl) and the concentration was measured over time. Compared with continuous systemic administration, subcutaneous bolus injection of the 0.05 mg/kg dose results in rapid elimination (FIG. 9) which, if repeated over time, for example daily, leads to an effect of peaks and valleys which is avoided by continuous administration.

2. Continuous Subcutaneous Administration is More Effective than the Subcutaneous Bolus Route in Inhibiting siRNA Target Gene Expression.

Mice were injected subcutaneously daily for 4 days with siTPS1-1 siRNA at a dose of 0.12 mg/kg/day, formulated in saline (154 mM NaCl), or the same siRNA formulated in the same solution but administered continuously for 4 days with an osmotic pump at the dose of 0.2 mg/kg/day. It is observed that the siTSP1-1 siRNA distributes in several organs at comparable levels after continuous or bolus subcutaneous administration (FIG. 10A). Administration of siTSP1-1 produces better tissue inhibition of TSP1 mRNA expression when siRNA is administered continuously than when administered as a bolus (FIG. 10B).

3. Continuous Subcutaneous Administration is More Effective than Bolus Subcutaneous Injection to Inhibit Tumor Growth

C4-2 cells were grafted into mice. Once the tumor was noted, the mice were treated by administration of siAR-1 siRNA formulated in saline (154 mM NaCl) at different doses or by vehicle. The siRNA was administered subcutaneously, either discontinuously, by daily injection, or continuously, by implantation of an osmotic pump for 1 month. Growth of C4-2 tumors in mice is not observed to be inhibited by subcutaneous bolus administration of siAR-1 siRNA at a dose of 0.12 mg/kg/day or even 1 mg/kg/day repeated daily (FIG. 11A), while it is inhibited when the same siRNA is administered subcutaneously continuously by implantation of an osmotic pump, delivering the dose of 0.2 mg/kg/day. Inhibition is not improved by increasing the administered dose by a factor of 10 (2 mg/kg/day) (FIG. 11B). Systemic administration of the same siRNA is therefore more effective in inhibiting tumor growth when this siRNA is administered continuously than discontinuously.

4. Inhibition of Bone Metastases from a Tumor

22RV1 cells were implanted in Nude mice. Once the tumor was detected, Alzet pumps administering 0.2 mg/kg/day of SiAR-1 siRNA formulated in saline (154 mM NaCl), or the vehicle alone were implanted for 3 weeks. At the end of treatment, the bones (tibia) were recovered to quantify siAR-1, and the mRNAs of human origin coding for the androgen receptor and the HPRT.

Continuously subcutaneous administration of siAR-1 siRNA to mice bearing 22RV1 human prostate tumors inhibits androgen receptor expression in the bones of mice (FIG. 12 left panel). This inhibition is accompanied by a decrease in the number of mice spontaneously developing bone metastases of these tumors and a decrease in the size of these tumors, evaluated by the level of expression of a human mRNA (HRPT) in the cells. bone (FIG. 12 right panel).

The continuous systemic administration of a siRNA therefore makes it possible to deliver it into the metastases of a cancer developing in the bone, to inhibit the expression of the target gene of the siRNA in the bone and to limit the implantation and/or the development of metastases.

### 5. Inhibition of Androgen Receptor Expression in the Prostate of Mice and Rats

Osmotic pumps delivering SiAR-1 siRNA formulated in saline (154 mM NaCl) were implanted subcutaneously in mice for 1 month or in rats for 2 weeks. This administration to mice at a dose greater than or equal to 0.2 mg/kg (FIG. 13) or to rats at a dose greater than or equal to 0.1 mg/kg (FIG. 14) effectively inhibits the protein expression of the androgen receptor. in the prostate of these animals.

6. Distribution of siRNA in Tissues in Monkeys

Adult male monkeys received for 4 weeks a continuous subcutaneous injection of siAR-1 formulated in saline (154 mM NaCl) at a dose of 5 mg/kg/day, at using an osmotic pump. The animals were sacrificed and siAR-1 quantified in different organs.

The results are shown in FIG. 15. It is found that siAR-1 siRNA distributes efficiently systemically in the various organs analyzed.

### 7. Inhibition of PSA Production in Monkeys

Prostate-specific antigen or PSA is detected in the serum of mature male monkeys, even in the absence of prostatic pathology.

Continuous subcutaneous administration of siAR-1 siRNA for 4 weeks at a dose of 5 mg/kg/day leads to a decrease in PSA expression in animal serum, below the detection limit of the ELISA test used. for the assay (FIG. 16). The continuous subcutaneous administration of a siRNA in monkeys thus makes it possible to distribute it systemically in numerous organs in which it exerts its inhibitory effect on gene expression.

### Example 7

Inhibition of TSP1 expression in glioblastoma by continuous intracerebral administration of siRNA formulated in saline solution (154 mM NaCl). Female nude mice were grafted orthotopically with U87 glioblastoma cells. The animals were implanted with an osmotic pump placed subcutaneously in the back, the output of the pump being connected to a catheter delivering in the brain, at a distance from the tumor, a control siRNA or the siRNA siTSP1-1 contained in the pump, at a rate of 2 mg/kg of brain weight/day. After 8 days, the mice were sacrificed, and the expression of TSP1 detected by immuno fluorescence on brain sections. The results are shown in FIG. 17.

There is a strong decrease in TSP1 expression in treated versus control animals. TSP1 is a protein that inhibits the formation of blood vessels (angiogenesis). In the treated animals, an increase in the density of the blood vessels detected on an adjacent section is observed by immuno-labeling of the CD31 antigen.

Continuous intracerebral administration of siRNA thus makes it possible to effectively inhibit the expression of the siRNA target gene in a tumor developing in this organ and to produce the expected biological effects therein.

### Example 8: An Acid Buffer Prevents the Degradation of a siRNA in the Presence of Cations

1. An siRNA is Degraded in the Presence of ZnCl2 in an Aqueous Solution Whose pH has been Adjusted to 6. Citrate Buffer at pH 6 Preserves siRNA

The siTSP1-1 siRNA degradation was measured after formulation of this siRNA under different conditions. The integrity of siTSP1-siRNA was verified by deposition on an acrylamide gel of an amount equivalent to 300 ng of siRNA from a siRNA solution that underwent the following treatments:
mixing (1:1, weight: weight) of siTSP1-1 and hexarelin; mixture (1:1 or 1:10, weight: weight in water) of oxidized siTSP1-1 and LDL; incubation for 4 h at 37′C.
mixing (1:1 or 1:10, weight: weight in water) of oxidized siTSP1-1 and LDL; incubation for 4 h at 37° C.
incubating for 1 hour at room temperature of siTSP1-1 in an aqueous solution of 0.164 mM ZnCl₂
incubating for 10 minutes, hourly or 6 hours at 37° C. of siTSP1-1 in a solution of 1 mM ZnCl₂ in a 10 mM Citrate buffer at pH 6.

It is found in FIG. 18 that siRNA is degraded when incubated in an aqueous solution of ZnCl₂. This degradation does not occur in citrate buffer at pH 6 containing up to 6 times more ZnCl₂. No degradation of siRNA is observed when mixed in water with oxidized LDL or hexarelin.

The presence of an acid pH buffer thus makes it possible to maintain the integrity of a siRNA in the presence of cations.

2. Formulation of an siRNA in an Aqueous Solution Containing ZnCl2 Reduces its Concentration in the Serum and its Distribution in the Tissues.

The siTSP1-1 siRNA formulated either in 154 mM NaCl or in water containing 0.165 mM ZnCl₂ and administered subcutaneously to mice at a dose of 0.12 mg/kg. The siRNA concentration measured in serum and tissues is reduced when the siRNA is formulated in an aqueous solution containing cations compared to the results obtained when the same siRNA is administered at the same dose and in the same way but formulated in saline solution. (154 mM NaCl) (FIG. 19).

### Example 9 Formulations Improving the Biodistribution of a siRNA In Vivo

1. The Serum and Tissue Concentration of a Systemically Administered siRNA is Increased when Formulated in Citrate Buffer and Even More in Citrate Buffer Containing Different Cations.

SiAR-1 siRNA at a dose of 0.12 mg/kg, formulated in different solutions, was administered subcutaneously to mice. The concentration of siAR-1 measured in the serum or organs of these different groups of mice was measured 20 minutes after injection and compared with that of the control group, consisting of animals having received the siRNA diluted in an aqueous solution containing 154 mM of NaCl (denoted NaCl).

In comparison with a formulation in a saline solution (154 mM NaCl), the formulation of SiAR-1 siRNA in 10 mM Citrate buffer pH 6 increases the concentration of this siRNA in the serum (FIG. 20A). This concentration is further increased in serum and tissues when 10 mM citrate buffer pH 6 is supplemented with ZnCl2, MgCl₂.

MgCl2, or a combination of these salts (FIG. 20B).

2. In Tissue and Tumor Serum, the Concentration of a Systemically Administered and Continuous siRNA is Increased when Formulated in a Citrate Buffer Containing Cations.

A mixture of 3 siRNAs, siAR-1, siTSP1-1 and siLuc, was administered subcutaneously continuously for 3 days to mice bearing murine mammary tumors 4T1 using an osmotic pump implanted subcutaneously. Each siRNA was administered at a dose of 2 mg/kg/day. The mixture was formulated in either a 154 mM NaCl solution or a 10 mM Citrate pH 6 buffer containing 10 mM MgCl₂. After 3 days, the concentration of each siRNA in serum, tumors and different organs was measured and the values measured when the siRNA had been formulated in citrate-MgCl₂ buffer were compared to the values measured with saline-formulated siRNA administration (154 mM NaCl) in the same fabric. The results reported in FIGS. 21A and 21B show that the formulation of siRNAs in citrate-MgCl₂ buffer increased by up to more than 250 their systemic distribution in tissues compared to their saline formulation.

### Example 10 Targeting a siRNA by Adding a CD36 Ligand

Mice received a subcutaneous injection of siAR-1 at a dose of 0.12 mg/kg formulated either in saline solution (154 mM NaCl) or 10 mM citrate buffer pH 6 containing hexarelin (siRNA ratio: Hexarelin; weight: 1:0.2), or in 10 mM citrate buffer pH6 containing oxidized LDL (siRNA ratio: oxidized LDL, weight: weight, 1:1). The concentration of siAR-1 was measured and related to the measured concentration when the siRNA was formulated in saline (154 mM NaCl) in the same tissue. The results observed in the serum are shown in FIG. 22A, in the prostate and the spleen in FIG. 22B. They show that the addition of hexarelin or oxidized LDL increases the concentration of siRNA in serum or tissues.

In another experiment, osmotic pumps were implanted in mice bearing 22RV1 tumors, the pumps delivering for 3 days continuously 2 mg/kg/day of siAR-1 formulated in saline solution (NaCl 154 mM), or 2 mg/kg./day of siAR-1 and 0.2 mg/kg/day of oxidized LDL formulated in 10 mM citrate buffer pH 6. In the latter case, the siRNA and the oxidized LDL were simply mixed in the citrate buffer, without additional manipulation. The concentrations of siAR-1 formulated in citrate buffer containing oxidized LDL measured in serum, tissues or tumors were related to the value measured in the same tissue when the siRNA was formulated in saline (154 mM NaCl). It can be seen in FIG. 23 that the presence of oxidized LDL increases the concentration of siAR-1 in serum tissues and tumors.

1-17. (canceled)

18. A Method for the prevention and/or treatment of a pathology comprising the administration of a composition comprising at least one siRNA, said siRNA hybridizing with a mRNA, coding or non-coding, of which it induces the degradation or of which it inhibits the translation, the expression of said mRNA or of the protein for which it encodes being involved in said pathology, said composition being formulated for a continuous systemic mode of administration.

19. The method according to claim 18, wherein said at least one siRNA is in a buffer solution at acidic pH, in particular in a citrate or histidine buffer.

20. The method according to claim 18, in which the at least one siRNA is in a buffer solution at acidic pH, added with inorganic or organic salts, in particular salt whose cation is chosen from polyamines, in particular chosen from spermine, spermidine or putrescine or in particular salt whose cation is chosen from metal cations, in particular chosen from salts of zinc, cobalt, copper, manganese, calcium, magnesium or iron, in particular manganese, zinc, magnesium, alone or in combination two to two or three to three.

21. The method according to claim 18, wherein said composition contains or does not contain an addressing agent, preferably containing an addressing agent not covalently coupled to siRNA.

22. The method according to claim 18, wherein said composition contains or does not contain an addressing agent, preferably containing an addressing agent not covalently coupled to siRNA, said addressing agent being a CD36 receptor ligand, said CD36 receptor ligand preferably being oxidized LDL, hexarelin, long chain fatty acid, or a mixture thereof two to two or three to three, said oxidized LDL being in a ratio weight:weight of 1 siRNA from 0.01 to 10 oxidized LDL and preferably from 0.1 to 1, or said hexarelin being in a ratio weight:weight of 1 siRNA from 0.01 to 10 hexarelin, preferably from 0.1 to 1.

23. The method according to claim 18, wherein said at least one siRNA is in a solution containing a vectorization agent.

24. The method according to claim 18, wherein said siRNA is in a solution containing no vectorization agent.

25. A device providing a means of continuous systemic mode of administration of a composition comprising at least one siRNA, said siRNA hybridizing with a mRNA, coding or non-coding, of which it induces the degradation or of which it inhibits the translation, the expression of said mRNA or of the protein for which it encodes being involved in said pathology, said composition being formulated for a continuous systemic mode of administration, said means of continuous systemic mode of administration being in particular an osmotic pump, a pump-syringe, an elastomeric pump, a peristaltic pump, an “intelligent” pump, a “patch” pump, or a polymeric matrix or a hydrogel, or any other biodegradable compound for slowly and continuously releasing the siRNA so that it is systemically distributed in the body.

26. The method according to claim 18, wherein said pathology is associated with expression of androgen receptor-encoding mRNA, Thrombospondin-1 (TSP1), FoxP3 transcription factor or Vascular Endothelial Growth Factor A (VEGF).

27. The method according to claim 18, wherein said at least one siRNA is one of the following siRNAs: siAR-1, siAR-1b, siAR-2, siAR-2b, siAR-3, siRNA 3b, siAR-4, siAR4b, siAR-5, siAR-5b, siVEGF-1, siVEGF-1b, siTSP1-1, siTSP1b-1, siTSP1-2, siTSP1-2b, siTSP1-3, siTSP1-3b, siTSP1-4, siTSP1-4b, siTSP1-5, siTSP1-5b, siFoxP3-1, siFoxP3-1b, siFoxP3-2, siFoxP3-2b, from SEQ ID NO: 1 to 52.

28. The method according to claim 18 for the prevention and/or treatment of a pathology associated with expression of the FoxP3 transcription factor comprising the administration of a medicament, said medicament having as active substance a siRNA chosen from the group consisting of: siFoxP3-1, siFoxP3-1b, siFoxP3-2 or siFoxP3-2b, of SEQ ID NOs 45-52, in association with a pharmaceutically acceptable carrier.

29. The method according to claim 18 for the prevention and/or treatment of a pathology associated with the expression of the androgen receptor, in particular for the prevention and/or treatment of prostate cancer, comprising the administration of a medicament, said medicament having as active substance a siRNA chosen from the group consisting of siAR-1, of SEQ ID NOs 1 and 2, in association with a pharmaceutically acceptable vehicle.

30. The method according to claim 18, wherein said at least one siRNA is devoid of chemical modifications or has chemical modifications.

31. The method according to claim 18, wherein said siRNA is used in combination with at least one anti-angiogenic agent or an anti-tumor agent or an immunotherapeutic agent or with a combination of these different classes of agents.

32. The method according to claim 18, wherein said systemic mode of administration is selected from the group consisting of or consisting of the subcutaneous, intraperitoneal, intravenous, intra-arterial, intracardiac, intramuscular, intradermal, intranasal, intravaginal, intrarectal, sublingual, oral, intrathecal, intraspinal, epidural, respiratory, cutaneous, transdermal, transmucosal.

33. The method according to claim 18, wherein said composition is formulated for a mode of administration at a therapeutically effective dose, and in particular from 0.005 mg/kg/day to 30 mg/kg/day, in particular from 0.01 mg/kg/day to 10 mg/kg/day and more particularly from 0.01 mg/kg/day to 2 mg/kg/day.

34. The method according to claim 18, wherein said pathology is a primary tumor, a metastatic tumor, or a pathology associated with the presence of suppressor or immunosuppressive cells, and is in particular a cancer of the anus, the appendix, mouth, bronchi and/or upper airways, bile duct, nasal and paranasal cavity, brain, heart, cervix, colon, body of the uterus, stomach, liver, salivary glands, throat, tongue, lips, nasopharynx, esophagus, bones, ovary, pancreas, parathyroid, penis, pleura, lung, androgen-independent prostate, rectum, kidney, breast, adrenals, testes, head and neck, thymus, thyroid, urethra, vagina, gall bladder, bladder, vulva, gastrointestinal cancer, lymphoma, melanoma or non-melanoma skin cancer, myeloma, sarcoma, leukemia, mesothelioma, cholangiocarcinoma, osteosarcoma, glioblastoma, astrocytoma, oligodendroglioma, chondrosarcoma, liposarcoma, rhabdomyosarcoma, or a pheochromocytoma, or the metastases of these cancers developing in other organs, and is in particular prostate cancer.


SEQUENCE LISTING (56 sequences)

SEQ ID NO: 1 - RNA, 21 nt, Artificial sequence
  Strand of a siRNA
  misc_feature (20)..(21): n is an overflowing deoxythymidine
gacucagcugccccauccann

SEQ ID NO: 2 - RNA, 21 nt, Artificial sequence
  Strand of a siRNA
  misc_feature (20)..(21): n is an overflowing deoxythymidine
uggauggggcagcugagucnn

SEQ ID NO: 3 - RNA, 19 nt, Artificial sequence
  Strand of a siRNA
gacucagcugccccaucca

SEQ ID NO: 4 - RNA, 19 nt, Artificial sequence
  Strand of a siRNA
uggauggggcagcugaguc

SEQ ID NO: 5 - RNA, 23 nt, Artificial sequence
  Strand of a siRNA
  misc_feature (22)..(23): n is an overflowing deoxythymidine
gacucagcugccccauccacgnn

SEQ ID NO: 6 - RNA, 23 nt, Artificial sequence
  Strand of a siRNA
  misc_feature (22)..(23): n is an overflowing deoxythymidine
cguggauggggcagcugagucnn

SEQ ID NO: 7 - RNA, 21 nt, Artificial sequence
  Strand of a siRNA
gacucagcugccccauccacg

SEQ ID NO: 8 - RNA, 21 nt, Artificial sequence
  Strand of a siRNA
cguggauggggcagcugaguc

SEQ ID NO: 9 - RNA, 21 nt, Artificial sequence
  Strand of a siRNA
  misc_feature (20)..(21): n is an overflowing deoxythymidine
uccccaagcccaucguagann

SEQ ID NO: 10 - RNA, 21 nt, Artificial sequence
  Strand of a siRNA
  misc_feature (20)..(21): n is an overflowing deoxythymidine
ucuacgaugggcuuggggann

SEQ ID NO: 11 - RNA, 19 nt, Artificial sequence
  Strand of a siRNA
uccccaagcccaucguaga

SEQ ID NO: 12 - RNA, 19 nt, Artificial sequence
  Strand of a siRNA
ucuacgaugggcuugggga

SEQ ID NO: 13 - RNA, 21 nt, Artificial sequence
  Strand of a siRNA
  misc_feature (20)..(21): n is an overflowing deoxythymidine
guaguugugaguaucaugann

SEQ ID NO: 14 - RNA, 21 nt, Artificial sequence
  Strand of a siRNA
  misc_feature (20)..(21): n is an overflowing deoxythymidine
ucaugauacucacaacuacnn

SEQ ID NO: 15 - RNA, 19 nt, Artificial sequence
  Strand of a siRNA
guaguugugaguaucauga

SEQ ID NO: 16 - RNA, 19 nt, Artificial sequence
  Strand of a siRNA
ucaugauacucacaacuac

SEQ ID NO: 17 - RNA, 21 nt, Artificial sequence
  Strand of a siRNA
  misc_feature (20)..(21): n is an overflowing deoxythymidine
gcaucaguucgcuuuugacnn

SEQ ID NO: 18 - RNA, 21 nt, Artificial sequence
  Strand of a siRNA
  misc_feature (20)..(21): n is an overflowing deoxythymidine
gucaaaagcgaacugaugcnn

SEQ ID NO: 19 - RNA, 19 nt, Artificial sequence
  Strand of a siRNA
gcaucaguucgcuuuugac

SEQ ID NO: 20 - RNA, 19 nt, Artificial sequence
  Strand of a siRNA
gucaaaagcgaacugaugc

SEQ ID NO: 21 - RNA, 23 nt, Artificial sequence
  Strand of a siRNA
  misc_feature (22)..(23): n is an overflowing deoxythymidine
augugaaugcagaccaaagaann

SEQ ID NO: 22 - RNA, 23 nt, Artificial sequence
  Strand of a siRNA
  misc_feature (22)..(23): n is an overflowing deoxythymidine
uucuuuggucugcauucacaunn

SEQ ID NO: 23 - RNA, 21 nt, Artificial sequence
  Strand of a siRNA
augugaaugcagaccaaagaa

SEQ ID NO: 24 - RNA, 21 nt, Artificial sequence
  Strand of a siRNA
uucuuuggucugcauucacau

SEQ ID NO: 25 - RNA, 21 nt, Artificial sequence
  Strand of a siRNA
  misc_feature (20)..(21): n is an overflowing deoxythymidine
ccuugacaacaacguggugnn

SEQ ID NO: 26 - RNA, 21 nt, Artificial sequence
  Strand of a siRNA
  misc_feature (20)..(21): n is an overflowing deoxythymidine
caccacguuguugucaaggnn

SEQ ID NO: 27 - RNA, 19 nt, Artificial sequence
  Strand of a siRNA
ccuugacaacaacguggug

SEQ ID NO: 28 - RNA, 19 nt, Artificial sequence
  Strand of a siRNA
caccacguuguugucaagg

SEQ ID NO: 29 - RNA, 21 nt, Artificial sequence
  Strand of a siRNA
  misc_feature (20)..(21): n is an overflowing deoxythymidine
uacccgagacgauuguaugnn

SEQ ID NO: 30 - RNA, 21 nt, Artificial sequence
  Strand of a siRNA
  misc_feature (20)..(21): n is an overflowing deoxythymidine
cauacaaucgucucggguann

SEQ ID NO: 31 - RNA, 19 nt, Artificial sequence
  Strand of a siRNA
uacccgagacgauuguaug

SEQ ID NO: 32 - RNA, 19 nt, Artificial sequence
  Strand of a siRNA
cauacaaucgucucgggua

SEQ ID NO: 33 - RNA, 21 nt, Artificial sequence
  Strand of a siRNA
  misc_feature (20)..(21): n is an overflowing deoxythymidine
gccagaacucgguuaccaunn

SEQ ID NO: 34 - RNA, 21 nt, Artificial sequence
  Strand of a siRNA
  misc_feature (20)..(21): n is an overflowing deoxythymidine
augguaaccgaguucuggcnn

SEQ ID NO: 35 - RNA, 19 nt, Artificial sequence
  Strand of a siRNA
gccagaacucgguuaccau

SEQ ID NO: 36 - RNA, 19 nt, Artificial sequence
  Strand of a siRNA
augguaaccgaguucuggc

SEQ ID NO: 37 - RNA, 21 nt, Artificial sequence
  Strand of a siRNA
  misc_feature (20)..(21): n is an overflowing deoxythymidine
ccaacaaacaggugugcaann

SEQ ID NO: 38 - RNA, 21 nt, Artificial sequence
  Strand of a siRNA
  misc_feature (20)..(21): n is an overflowing deoxythymidine
uugcacaccuguuuguuggnn

SEQ ID NO: 39 - RNA, 19 nt, Artificial sequence
  Strand of a siRNA
ccaacaaacaggugugcaa

SEQ ID NO: 40 - RNA, 19 nt, Artificial sequence
  Strand of a siRNA
uugcacaccuguuuguugg

SEQ ID NO: 41 - RNA, 21 nt, Artificial sequence
  Strand of a siRNA
  misc_feature (20)..(21): n is an overflowing deoxythymidine
gcaacuaccugggucacuann

SEQ ID NO: 42 - RNA, 21 nt, Artificial sequence
  Strand of a siRNA
  misc_feature (20)..(21): n is an overflowing deoxythymidine
uagugacccagguaguugcnn

SEQ ID NO: 43 - RNA, 19 nt, Artificial sequence
  Strand of a siRNA
gcaacuaccugggucacua

SEQ ID NO: 44 - RNA, 19 nt, Artificial sequence
  Strand of a siRNA
uagugacccagguaguugc

SEQ ID NO: 45 - RNA, 21 nt, Artificial sequence
  Strand of a siRNA
  misc_feature (20)..(21): n is an overflowing deoxythymidine
cacaacauggacuacuucann

SEQ ID NO: 46 - RNA, 21 nt, Artificial sequence
  Strand of a siRNA
  misc_feature (20)..(21): n is an overflowing deoxythymidine
ugaaguaguccauguugugnn

SEQ ID NO: 47 - RNA, 19 nt, Artificial sequence
  Strand of a siRNA
cacaacauggacuacuuca

SEQ ID NO: 48 - RNA, 19 nt, Artificial sequence
  Strand of a siRNA
ugaaguaguccauguugug

SEQ ID NO: 49 - RNA, 21 nt, Artificial sequence
  Strand of a siRNA
  misc_feature (20)..(21): n is an overflowing deoxythymidine
cauggacuacuucaaguucnn

SEQ ID NO: 50 - RNA, 21 nt, Artificial sequence
  Strand of a siRNA
  misc_feature (20)..(21): n is an overflowing deoxythymidine
gaacuugaaguaguccaugnn

SEQ ID NO: 51 - RNA, 19 nt, Artificial sequence
  Strand of a siRNA
cauggacuacuucaaguuc

SEQ ID NO: 52 - RNA, 19 nt, Artificial sequence
  Strand of a siRNA
gaacuugaaguaguccaug

SEQ ID NO: 53 - RNA, 23 nt, Artificial sequence
  Strand of a siRNA
ggguuaugucuauguucauucuu

SEQ ID NO: 54 - RNA, 23 nt, Artificial sequence
  Strand of a siRNA
ggaauggccugugcuuucucauu

SEQ ID NO: 55 - RNA, 21 nt, Artificial sequence
  Strand of a siRNA
  misc_feature (20)..(21): n is an overflowing deoxythymidine
uagcaaugacgaaugcguann

SEQ ID NO: 56 - RNA, 21 nt, Artificial sequence
  Strand of a siRNA
  misc_feature (20)..(21): n is an overflowing deoxythymidine
uacgcauucgucauugcuann